SEQUENCE LISTING (18 sequences)

SEQ ID NO: 1 - DNA, 16 bp, synthetic construct
  source 1..16: mol_type = other DNA
  modified_base 1..16: mod_base = OTHER; note = phosphorothioate backbone
  modified_base 1..3: mod_base = OTHER; note = locked nucleic acids
  modified_base 14..16: mod_base = OTHER; note = locked nucleic acids
gtcgtgtcaagtcatc

SEQ ID NO: 2 - DNA, 16 bp, synthetic construct
  source 1..16: mol_type = other DNA
  modified_base 1..16: mod_base = OTHER; note = phosphorothioate backbone
  modified_base 1..3: mod_base = OTHER; note = locked nucleic acids
  modified_base 14..16: mod_base = OTHER; note = locked nucleic acids
ccatagaagaattact

SEQ ID NO: 3 - DNA, 16 bp, synthetic construct
  source 1..16: mol_type = other DNA
  modified_base 1..16: mod_base = OTHER; note = phosphorothioate backbone
  modified_base 1..3: mod_base = OTHER; note = locked nucleic acids
  modified_base 14..16: mod_base = OTHER; note = locked nucleic acids
gaagtcgtgtcaagtc

SEQ ID NO: 4 - DNA, 16 bp, synthetic construct
  source 1..16: mol_type = other DNA
  modified_base 1..16: mod_base = OTHER; note = phosphorothioate backbone
  modified_base 1..3: mod_base = OTHER; note = locked nucleic acids
  modified_base 14..16: mod_base = OTHER; note = locked nucleic acids
tgtcgtccactctaat

SEQ ID NO: 5 - DNA, 15 bp, synthetic construct
  source 1..15: mol_type = other DNA
  modified_base 1..15: mod_base = OTHER; note = phosphorothioate backbone
  modified_base 1..3: mod_base = OTHER; note = locked nucleic acids
  modified_base 13..15: mod_base = OTHER; note = locked nucleic acids
aacacgtctatacgc

SEQ ID NO: 6 - DNA, 19 bp, synthetic construct
  source 1..19: mol_type = other DNA
tattctggcccatcgtggc

SEQ ID NO: 7 - DNA, 20 bp, synthetic construct
  source 1..20: mol_type = other DNA
ctgaagcagagctcgaccag

SEQ ID NO: 8 - DNA, 25 bp, synthetic construct
  misc_feature 1..23: note = RNA
  misc_feature 24..25: note = DNA
  source 1..25: mol_type = other DNA
cactctgagatagaatctaattgga

SEQ ID NO: 9 - RNA, 27 nt, synthetic construct
  source 1..27: mol_type = other RNA
tccaattagattctatctcagagtgaa

SEQ ID NO: 10 - RNA, 21 nt, synthetic construct
  source 1..21: mol_type = other RNA
atgacctatgaattgacagac

SEQ ID NO: 11 - RNA, 21 nt, synthetic construct
  source 1..21: mol_type = other RNA
ctgacctatgaattgacagcc

SEQ ID NO: 12 - DNA, 18 bp, synthetic construct
  source 1..18: mol_type = other DNA
gctgagatatctaggtca

SEQ ID NO: 13 - DNA, 22 bp, synthetic construct
  source 1..22: mol_type = other DNA
tcgatgacctagatatctcagc

SEQ ID NO: 14 - DNA, 18 bp, synthetic construct
  source 1..18: mol_type = other DNA
cttagcatatgcacgacg

SEQ ID NO: 15 - DNA, 22 bp, synthetic construct
  source 1..22: mol_type = other DNA
tcgacgtcgtgcatatgctaag

SEQ ID NO: 16 - RNA, 22 nt, synthetic construct
  source 1..22: mol_type = other RNA
tcaccgggtgtaaatcagcttg

SEQ ID NO: 17 - DNA, 16 bp, synthetic construct
  source 1..16: mol_type = other DNA
  modified_base 1..16: mod_base = OTHER; note = phosphothioate backbone
  modified_base 1..3: mod_base = OTHER; note = locked nucleic acids
  modified_base 14..16: mod_base = OTHER; note = locked nucleic acids
acattggaaggatcaa

SEQ ID NO: 18 - DNA, 2472 bp, synthetic construct
  source 1..2472: mol_type = other DNA
gctttttttttttttttggtctcctaacatctattcaagcttttatttcattttgattctcagttctgtttctcaaatttcagatcaaggatgctttagaagtagaagtagcctattacaacattcatcttatgggctccgggaaaacaccgggtggggccagaaagctgaaatgatttccccaagatgagacagagagtctcagagccaacccatggttcctgattctaagactagtgctcttgctattaaaaaatattcaactgtttctttaactgattagcagaatttatttcaagactgctttgcctaaaattatctataagacagttacataagcctttaactctaatttcttaataccaatccaggatcttttcagttactggagcttcactgtactgctatctttagggtacacgccaataaaaaatatttgtcagaggttatttacaagaagttcttttgctactttggtaataaattattcttagatccttagatcatacgcagtatgggccaacccttagagcaattatcacaaccacaaactcttctaaaaactgacatagcacctgctgagcaagactgccttaggaaattccttatccattctttggctacaattccctactacattttggcctcggctaattggaccagagggtgaacacctgaccaaaggcaaacattccattggctggtcagcatcctattaggaggcctgaaatgaaagctttgaccaataaaaatcaagataacaggtccaattagattctatctcagagtgaaaaagagtcaaagtcaactagtttctaacaaatgctgtgatccacacttcagattggagtctgtgaaatatgcaccaacttcaagacaattccgtggtaaaactggaaaaaattagtcattaagacattagccaggatgttagtggtatgagaggatataaacatgcagccttgaaattaaaatacatagcctttaggctttgacctagatatctcagcaaagtgaacaaagaatagcattaaaagtaaataaaagattgaggcaggaggatagcttgaagtcaggagtttgagattggcctgtgcagtacagcgagacctcttctctacataaaaatgttaaaattagatacaaatataagcccgtaaataatctttgatcttaaaatttctaatttggaaaagtgtcatttgtatggtgttctatattattaatggtttcaaactttaatcagagatatctttccttttcaggcaaagcacacaccatgaaagatactgctaaaagtttttaatattgccagtaatattatcaaggtaattaaagggcttgtaataatattttgtaataatatctgaatatcttagtgattaaacttggaaaagccaaactagatagagaatgacactgagaagagtagatagcacagggtccaaggtcaggcgtggttcaacaattgtccatagaaagtctttgttttgatcaggatatatgagttggaaaatttcgtgatcgtggatgaagttttctttgtactttcatgaaaaactaccatttaaaatttggcagatgtttaaaaatgatgatttttttaaaactaccatactagacaatgtcatttgtaaatcaatgtgtaattaaaacattttgaaaagatgcccataccagacctccagattttctccacatttgagtaacttggaatttgattttcttcatagcctgttagccattttgactgaaacaggaccacaccaaaggcaaaggctggttgtttcagtccagagatgtgcctccatagaagaattactttgaacaaatcttaatagattttaggaaaatattttaaaatcaactgttggagaatttaactattttcagatgaacagtttattgcttgattgtttttcccttcttattcttccatgtggttttatgcagaagtgatctggttgtaaaatatgaagagatggcaataaactcatttggcattatttggatcaagcagctggttgacttaaatcagttatatcacttctgtccacctccatgcgaagcactggtccagaccaccatctccctcacctgggttactacagcagctcccagctggtctctctgctcctcttctcacttctccaatctcagaaaggactggcctgaagcagagctcgaccaggacagcttcctcccaccacacaccagtccatctgtttctcatctgctgaccatgccacgatgggccagaataggattcaggaggcttctgaagtcgtgtcaagtcatctttcctcagctggatgactgcgatagcctcctactggtttcctgtcgtccactctaatctctttcccacagccacagtgaactttgtccaaaggaaatcagattgtgtactcccctgcttaaaaccttccaatggttttgcattgcactctaaaaaaaaaaaaaatctaactc

(19) United States
(12) Patent Application Publication
(10) Pub. No.: US 2026/0275335 A1
(43) Pub. Date: Sep. 17, 2026

(54) Methods and compositions targeting LINC01929 to increase antigen presentation

(71) Applicant: Oncolinc Therapeutics Inc, Halifax (CA)

(72) Inventors: Jaganathan Venkatesh, Halifax (CA); Marie-Claire D Wasson, Halifax (CA); Raj Pranap Arun, Halifax (CA); Hannah F Cahill, Waverley (CA); Bakhmala Khan, Halifax (CA); Paola Marcato, Hubley (CA)

(21) Appl. No.: 19/079,632

(22) Filed: Mar. 14, 2025

(51) Int. Cl. C12N 15/113; A61K 45/06; A61P 35/00; C12Q 1/6886

(52) U.S. Cl. CPC C12N 15/113; A61K 45/06; A61P 35/00; C12Q 1/6886; C12N 2310/113; C12N 2310/14; C12N 2310/3231; C12Q 2600/106

(57) ABSTRACT

Among other things, in general, specific anti-cancer immune cell responses associated with enhanced T cell killing (including increased antigen presentation, production of tumor-associated antigens by cancer cells, and increased PD-L1) is achieved by targeting a specific long non-coding RNA (lncRNA), the ENST00000592405.3 transcript, such as with, e.g., antisense oligonucleotides or siRNA.

### REFERENCE TO AN ELECTRONIC SEQUENCE LISTING AND A TABLE

The contents of an XML electronic sequence listing (6805_0001US_SEQ_LIST.xml; 29,642 bytes; created Mar. 12, 2025) is herein incorporated by reference in its entirety.

The contents of Table 2, a “large table,” provided in ASCII format (Table_2.txt; 453,627 bytes; created Mar. 4, 2025) is herein incorporated by reference in its entirety.

LENGTHY TABLES
The patent application contains a lengthy table section. A copy of the table is available in electronic form from the USPTO web site (https://seqdata.uspto.gov/docdetail?docId=US20260275335A1). An electronic copy of the table will also be available from the USPTO upon request and payment of the fee set forth in 37 CFR 1.19(b)(3).

### BACKGROUND OF THE INVENTION

Cancer development and progression are largely influenced by T cells in the tumor microenvironment. T cells have the capacity to kill cancer, so to avoid destruction by the immune system, cancer cells have mechanisms in place to avoid immune cell detection. Cancer cells are resistant to T cell killing because of impaired antigen presentation.

High abundance of T cells and increased expression of the programmed death-ligand 1 protein 1 (PD-L1) are associated with better cancer outcomes and treatment efficacy with immune checkpoint inhibitors (ICIs), like pembrolizumab. ICIs inhibit tumor growth and metastasis, supporting anti-tumor T cell activity by blocking the interaction between the programmed cell death protein 1 (PD-1) receptor on T cells and its ligand PD-L1 on cancer cells. To successfully mediate these effects, ICIs require a “hot” tumor microenvironment: one that contains abundant cytotoxic T cells, a high tumor mutational burden leading to increased presentation of neoantigens, and the presence of PD-L1+ cells. ICIs are now used to treat a variety of cancers, including lung, melanoma, bladder, cervical, liver, stomach, and the aggressive triple-negative breast cancer (TNBC) subtype.

Even tumors with a more immune-active microenvironment, with abundant T cells (“hot” tumors) often do not respond to ICI because immune evasion and suppressive mechanisms outweigh local anti-tumor immunity. As a general mechanism of cancer development and progression and avoidance of immune surveillance, cancers successfully evade immune cell killing through downregulation of the antigen presentation machinery, which includes the major histocompatibility complex class I (MHC I, or human leukocyte antigen class I (HLA I) in humans), immunoproteasomes (generate peptides compatible with MHC I), and transporter associated with antigen processing (TAP, that load the peptides onto MHC I). This results in the decreased production and presentation of peptides from tumor-associated antigens and therefore a lack of recognition of the cancer cells as foreign by T cells.

Several approaches have been explored to enhance antigen presentation in cancer cells. The use of epigenetic modulators like histone deacetylase inhibitors and DNA methyltransferase inhibitors has been investigated to enhance the maturation of dendritic cells and increase tumor antigen presentation. Research has shown that cancer cells utilize an evolutionarily conserved function of the polycomb repressive complex 2 to silence the MHC I antigen processing and presentation pathway. Targeting this mechanism could potentially restore antigen presentation in cancer cells. Strategies to increase the number of mutations in cancer cells have been explored to generate more neoantigens, potentially enhancing tumor immunogenicity.

There are many drugs or stimulants that could induce antigen presentations in cancer cells. For example, chemotherapies (e.g., bleomycin, 5-fluorouracil, doxorubicin, gemcitabine) can induce immunogenic cell death, enhance antigen presentation and activate T cell antitumor activity. Targeted therapies (e.g., birinapant) can also augment antigen presentation and T cell killing of cancer cells. Cytokines like interferon-gamma (IFN-v) induce antigen presentation, and there are many indirect ways to induce IFN-γ production which leads to increased antigen presentation. For example, viral (e.g. influenza) and bacterial (e.g., mycobacterium) infections and vaccines (e.g., BCG vaccine) can induce IFN-γ production by immune cells.

Despite these advancements, there remains a significant need for more effective and broadly applicable methods to enhance antigen presentation in cancer cells. The application of many of these prior methods to induce antigen presentation and anti-cancer activity and treat cancer in patients is hindered by the lack of specificity for targeting the cancer cells.

ASOs and siRNAs are both methods of targeting specific RNA species (most typically mRNA) to cause the degradation of RNA and inhibit its translation into protein. ASOs have applications in gene therapy, treatment of genetic diseases, cancer therapy, and RNA-based drugs. For example, the ASO TASO (TGF-β2 targeting anti-sense oligonucleotide)-001 is in clinical trials in combination with recombinant interleukin-2 (Aldesleukin) for advanced or metastatic solid tumors.

A specific subclass of ASOs, known as LNA gapmers, is designed to further optimize therapeutic efficacy (U.S. Pat. Nos. 6,268,490, 6,770,748, 6,639,059, 6,734,291; hereby incorporated by reference). LNA gapmers are polynucleotides with the 5′ and 3′ ends containing 2-4 locked nucleic acid (LNA) modifications. These LNA-modified “wings” increase nuclease resistance and target binding affinity. The central section of the gapmer forms a “gap” that allows RNase H to bind and cleave the target RNA. The entire oligonucleotide usually incorporates a backbone, e.g., phosphorothioate (PS), to enhance stability. LNA gapmers can be potent and selective tools in RNA-targeting therapies.

Through the conserved biology mechanism known as RNA interference (RNAi), synthesized double stranded RNA (dsRNA) can also be used to reduce/silence expression of specific genes. DsRNA can also degrade target RNA in various organisms, including plants, fruit flies, and mammals.

Small interfering RNAs (siRNAs) are dsRNA molecules that can have different modifications, including chemical modifications (e.g., 2′-O-methylation, 2′-fluorination) to improve stability and specificity, and conjugation with lipids or polymers to enhance delivery. These modifications have enabled siRNA to target a broader range of diseases, including cancer, genetic disorders, infectious diseases, and neurological disorders. Approved siRNA-based drugs include Givosiran (GIVLAARI™) which targets the aminolevulinic acid synthase 1 (ALAS1) mRNA to treat acute hepatic porphyria. SiRNA are also in clinical trials to treat cancer, such as the EphA2 siRNA (targeting the EPH receptor A2 mRNA) for treating patients with advanced or recurrent solid tumors (NCT01591356).

Despite their abundance, which is comparable in number to protein-coding genes, long non-coding RNAs (lncRNAs) remain relatively understudied. Through sequence complementarity and secondary structures, lncRNAs interact with RNA, chromatin and protein, and can function as competitive endogenous RNA (ceRNAs) with microRNAs (miRNAs) to modulate gene expression and cell signaling pathways. This versatility positions lncRNAs as key players in cancer biology and potential therapeutic targets. For instance, lncRNA capable of inducing MHC I and immunogenicity of tumor (LIMIT) upregulates MHC I and its expression positively correlated with tumor-infiltrating lymphocytes (TILs) and ICI response in melanoma. Metastasis-associated lung adenocarcinoma transcript 1 (MALAT1) regulates PD-L1 expression in non-small cell lung cancers, while HOTAIR induces PD-L1 expression in laryngeal squamous cell carcinoma and glioma. Epigenetically induced lincRNA 1 (EPIC1) suppresses IFN-γ cell signaling responses and the expression of genes involved in antigen presentation.

Yet identifying novel lncRNAs that play a functional in suppressing antigen presentation is challenging. This difficulty arises since the function of lncRNAs cannot be predicted from their sequence. Even when a lncRNA has been identified as possibly playing a role in tumor immune response, the design of therapeutics against a specific long non-coding RNA is still very difficult, particularly given the technical challenges of identifying a suitably compact target region of RNA for a given lncRNA. Moreover, the desired anti-cancer effect or combination of anti-cancer effects vary considerably and unpredictably depending on which region (or specific transcript) of a given lncRNA is targeted, which further adds to the difficulty of therapeutic design in this area.

There remains a need for lncRNA-targeting therapeutics that can overturn suppressed antigen presentation by cancer cells.

### SUMMARY OF THE INVENTION

Disclosed herein are methods and compositions to increase antigen presentation and induce specific anti-cancer cell immune cell responses associated with enhanced T cell killing of cancer cells. These responses include increased HLA I expression on cancer cells, proteasomal activity, activation of extrinsic apoptosis pathways, increased epitope presentation and the increase production and presentation of tumor-associated antigenic peptides. The methods and compositions increase antigen presentation by cancer cells, and thereby T cell activation against cancer cells, by using antisense oligonucleotides (ASOs) or small interfering RNA (siRNA) targeting expression of a transcript that is highly expressed in cancer cells and has shown particular utility in suppressing antigen presentation and anti-tumor immune activity in cancer cells.

More specifically, disclosed herein are methods to increase antigen presentation by cancer cells, and thereby T cell activation against cancer cells, by supplying antisense oligonucleotides or siRNA that target expression of the 2472 nucleotide transcript (ENST00000592405.3) of the long non-coding RNA (lncRNA) LINC01929. This is unexpected and surprising because the expression of over a thousand lncRNA genes, such as LINC01977, are associated with cold tumors with low T cell infiltration and yet in general targeting these lncRNAs does not induce antigen presentation in cancer cells because it is expected to require a specific gene expression reprogramming of genes that encode the antigen presentation machinery. In contrast, T cell infiltration of tumors is largely determined by expression of cytokines and chemokines which attract T cells to a tissue site (e.g. CCL2, CCL3, CCL4, CCL19, CXCL9, CXCL10, and CXCL11).

The development of novel approaches that can overcome antigen presentation downregulation mechanisms employed by different cancer types represents a significant advancement in the field of cancer immunotherapy and is distinct from immunotherapies which regulate immune cell infiltration or act as ICI.

In general, in an aspect, a composition is disclosed that includes a synthetic nucleic acid encoding a sequence capable of binding to a target sequence, where the complementary target sequence is at least 80% but less than 100% complementary to LINC01929 transcript ENST00000592405.3. Implementations may include one or more of the following. The nucleic acid is an antisense oligonucleotide. The nucleic acid is a double-stranded RNA. The nucleic acid has one or modifications to its RNA or DNA or both, such as locked nucleic acid (LNA), morpholino, peptide nucleic acid (PNA), unlocked nucleic acid (UNA), threose nucleic acid (TNA), glycol nucleic acid (GNA), FANA (Fluoro-arabino nucleic acid), or modified RNA selected from 2′-O-methyl(2′Ome), 2′-fluoro (2′F), 2′-O-methoxyethyl(2′OMOE), phosphorothioate (PS), cholesterol conjugation, GalNAc conjugation, polyethylene glycol (PEG) conjugation, unlocked nucleic acid (UNA), bridged nucleic acid (BNA), or 2′-4′ constrained ethyl(2′4′CE). The composition is an antisense oligonucleotide having a sequence selected from SEQ ID NOs: 1, 2, 3, 4, and 18 or some effective portion of SEQ ID NO:18. The composition includes locked nucleic acid. The composition is a short interfering RNA having a sequence selected from SEQ ID NO:8 and SEQ ID NO:9, and 18 or some effective portion of SEQ ID NO:18. The composition further includes a pharmaceutically acceptable carrier.

In general, in an aspect, an expression construct or vector is disclosed, where the construct or vector encodes the sequence of a synthetic nucleic acid that encodes a sequence capable of binding to a target sequence, where the complementary target sequence is at least 80% but less than 100% complementary to LINC01929 transcript ENST00000592405.3.

In general, in an aspect, a method for treating a cancer in a subject in need thereof is disclosed. The method includes administering to the subject at least an effective amount of a therapeutic comprising a synthetic nucleic acid that encodes a sequence capable of binding to a target sequence, where the complementary target sequence is at least 80% but less than 100% complementary to LINC01929 transcript ENST00000592405.3. Implementations may include one or more of the following. A standard of care for the cancer is administered before, during, or after the cancer therapy described herein. The standard of care includes administration of an immune checkpoint inhibitor. Prior to any other steps, applicability of LINC01929 therapy is identified using previously collected specimens of cancer subjects, the method including mixing labeled probes that are complementary to the second longest LINC01929 transcript (ENST00000592405.3) into the specimen and quantifying the result using an applicable sequencing or amplification paradigm (optionally including qPCR or RNA sequencing) to confirm applicability of LINC01929 transcript ENST00000592405.3 targeting therapy, wherein the therapeutic is administered when the expression level of LINC01929 is greater than a predetermined reference level. Cell surface expression of HLA I/MHC I is increased in cancer cells of the subject. Proteosome activity, optionally including immunoproteasome activity, is induced in the subject. Cancer cells of the subject have HLA I/MHC I presentation such that the number or diversity of immunostimulatory peptides, inclusive of tumor-associated antigens or neoantigens, are increased. T cells in the subject increase killing of cancer cells of the subject. Cancer cells of the subject are induced to produce PD-L1 on their surfaces. Cancer cells become more susceptible to death by the checkpoint inhibitor agent. The cancer is selected from a primary or metastatic brain tumor, a breast cancer, a neuroendocrine tumor, a melanoma, a pancreatic cancer, a prostate cancer, a head and neck cancer, an ovarian cancer, a lung cancer, a bladder cancer, a kidney cancer, a liver cancer, a vaginal cancer, a urogenital cancer, a gastric cancer, a colorectal cancer, a cervical cancer, a liposarcoma, an angiosarcoma, a rhabdomyosarcoma, a choriocarcinoma, a retinoblastoma, a multiple myeloma, a primary or metastatic breast cancer, an inflammatory breast cancer, a Ductal Carcinoma In Situ (DCIS), invasive ductal carcinoma (IDC) including IDC Type: Tubular Carcinoma of the Breast, IDC Type: Medullary Carcinoma of the Breast, IDC Type: Mucinous Carcinoma of the Breast, IDC Type: Papillary Carcinoma of the Breast, IDC Type: Cribriform Carcinoma of the Breast, as well as Invasive Lobular Carcinoma (ILC), Lobular Carcinoma In Situ (LCIS), Male Breast Cancer, Molecular Subtypes of Breast Cancer, Paget's Disease of the Nipple, Phyllodes Tumors of the Breast, Triple Negative Breast Cancer, and a Metastatic Breast Cancer.

### BRIEF DESCRIPTION OF DRAWINGS

FIG. 1 shows LINC01929 tumor tissue enrichment as captured in the Cancer Genome Atlas Program (TCGA) cohorts. The log 2 fold change in lncRNA expression (RPKM RNA-seq) between tumor and normal-adjacent tissue is shown for the 13 TANRIC datasets with normal-adjacent samples, as well as for the ER+/PR+, HER2+, and triple negative breast cancer (TNBC) subtypes from the TCGA-BRCA dataset. Error bars represent 95% confidence intervals, and * indicates an adjusted p-value<0.05.

FIG. 2 shows TCGA cohorts where high LINC01929 RNA levels had significant associations with worse patient survival.

FIG. 3 reports the number of lncRNAs associated with oncogenic properties in the TCGA-BRCA cohort. The association between lncRNA expression (RPKM RNA-seq) and TIL proportions, patient survival, and tumor tissue enrichment in breast cancer were analyzed using the TCGA-BRCA and corresponding TANRIC datasets. LncRNAs that were negatively correlated with TIL proportions (Pearson coefficient<0, p-value<0.05, N=763), associated with worse patient survival (hazard ratio (HR)>1, p-value<0.05, N=837), and enriched in tumor tissue compared to adjacent normal tissue (log₂FC>0, adjusted p-value<0.05, N=837 tumor samples, N=105 normal-adjacent samples) were identified.

FIG. 4 shows the tumor cell type proportions estimated using CIBERSORTx in TCGA-BRCA tumors categorized by high (N=261) and low (N=247) LINC01929 expression levels, based on the top and bottom 33rd percentiles. Statistical significance was evaluated using the Mann-Whitney test, with box and whisker plots generated using the Tukey method.

FIG. 5 shows the tumor cell type proportions estimated using CIBERSORTx in TCGA-BRCA tumors categorized by high (N=261) and low (N=247) LINC01977 expression levels, based on the top and bottom 33rd percentiles. Statistical significance was evaluated using the Mann-Whitney test, with box and whisker plots generated using the Tukey method.

FIG. 6 pearson correlation coefficients (R) between CD3+ and CD3+CD8+ T cell proportions and LINC01929 expression.

FIG. 7A shows the genomic location of the LINC01929 gene and its eleven transcripts with differing exons. The position targeted by various primers and therapeutics (ASO and siRNAs) described herein is shown by labeled vertical lines.

FIG. 7B summarizes which of the 11 transcripts of LINC01929 are targeted by the ASOs and siRNA.

FIG. 8 illustrates effects of ASOs described herein (ASO1-ASO4 corresponding to SEQ ID NOs: 1-4), which reduce the level of some of the LINC01929 transcripts (inclusive of ENST0000592405.3, the reverse complement of which is presented for reference as SEQ ID NO: 18) compared to cells treated with negative control ASO (SEQ ID NO: 5) by quantitative polymerase chain reaction (qPCR) analysis of LINC01929 expression 48 hours after post treatment (N=4, analyzed using primers SEQ ID NO:6 and SEQ ID NO:7). Notably the longest transcript ENST00000589125.5 is not targeted by ASO1, 3 and 4 (FIG. 7A, FIG. 7B).

FIG. 9 illustrates effects of siRNAs described herein (i.e., SEQ ID NO: 8 annealed to SEQ ID NO: 9) which targets only the two longest LINC01929 transcripts (described as ENST00000589125.5 and ENST0000592405.3) compared to cells treated with negative control (scrambled forward annealed to scrambled reverse) siRNA by qPCR analysis of LINC01929 expression 48 hours after post treatment using primers specific to LINC01929 (N=4, analyzed using primers SEQ ID NO:6 and SEQ ID NO:7). Statistical significance was assessed using a one-way ANOVA followed by Dunnett's multiple comparisons test. Error bars represent the standard deviation.

FIG. 10 shows log 2 fold changes in gene expression in MCF7 (left) and MDA-MB-231 (right) cells, as determined by an Affymetrix 2.0ST gene array (N=3) following treatment of LINC01929-targeting ASO1 (SEQ ID NO: 1, as shown by qpcr in FIG. 8). Treatment reduces the level of the second longest LINC01929 transcript (ENST0000592405.3) compared to ASO negative control treatments (SEQ ID NO: 5, N=3). A significance threshold of p<0.05 and fold-change cutoffs of ±1.2311 were applied. Labeled genes represent those of particular interest.

FIG. 11 shows the number of genes identified in the gene array in FIG. 10 and upregulated or downregulated in MCF7 and MDA-MB-231 cells which met the applied cutoffs described in FIG. 10.

FIG. 12 shows the qPCR validation of select differentially expressed genes from the gene array analysis in FIG. 10. Data represents relative expression of genes treated with LINC01929-targeting ASO1 (SEQ ID NO: 1) and ASO2 (SEQ ID NO: 2) compared to ASO negative control treatments (N=4, SEQ ID NO: 5). Statistical significance was determined using a one-way ANOVA followed by Dunnett's multiple comparisons test. Error bars represent the standard deviation.

FIG. 13 shows different example ASOs, ASO3 (SEQ ID NO: 3) and ASO4 (SEQ ID NO: 4) that also target LINC01929. Notably, ASO3 and ASO4 also affect the expression of key immune response genes and cancer promoting genes identified from the gene array analysis in FIG. 10. Data represents relative expression of genes treated with LINC01929-targeting ASO3 and ASO4 and compared to ASO negative control treatments (N=4, SEQ ID NO: 5). Statistical significance was determined using a one-way ANOVA followed by Dunnett's multiple comparisons test. Error bars represent the standard deviation.

FIG. 14 shows an siRNA that targets two LINC01929 transcripts specifically (i.e., selective for the two longest LINC01929 transcripts (ENST00000589125.5 and ENST00000592405.3)) also affects the expression of key immune response genes and cancer promoting genes identified from the gene array analysis in FIG. 11. Data represents relative expression of genes treated with LINC01929-targeting siRNA (SEQ ID NO: 8 and SEQ ID NO: 9) compared to negative control siRNA treatments (N=4). Statistical significance was determined using a t-test. Error bars represent the standard deviation. Notably, ASO1, ASO3 and ASO4 only target ENST00000592405.3 and not ENST00000589125.5 (FIG. 7). Together this siRNA and ASO data demonstrate that targeting ENST00000592405.3 is the critical transcript inducing expressing of key immune genes. ENST00000592405.3 is unique among the 11 transcripts in that it is targeted by all four ASO sequences and the siRNA sequence. The ASOs and siRNA target sequences present on the distinct exons, that together are only fully present in ENST00000592405.3 (FIG. 7B).

FIG. 15 shows that treatment with mimics of miRNAs previously reported as regulated by LINC01929 (miR-1179, miR-137-3p, and miR-6875-5p) do not affect the expression of a subset of key immune response genes and cancer promoting genes identified from the gene array analysis in FIG. 10. Statistical significance was determined using a one-way ANOVA followed by Dunnett's multiple comparisons test. Error bars represent the standard deviation.

FIG. 16 shows differentially expressed miRNAs upon treatment of MCF7 and MDA-MB-231 cells with LINC01929-targeting ASO1 (SEQ ID NO: 1) compared to ASO negative control treatments for 48 h (N=3, SEQ ID NO: 5). Log₂ fold-change of miRNA levels was determined by small RNA sequencing in MCF7 (left) and MDA-MB-231 (right) cells. The previously reported miR-1179, miR-137-3p, and miR-6875-5p (labeled here for emphasis) are not among the differentially expressed miRNAs. A significance threshold of p<0.05 and fold-change of +1.2311 was applied. The upper quadrants display the number of significantly upregulated or downregulated miRNAs.

FIG. 17A shows curated biological processes enriched in genes upregulated in MCF7 and MDA-MB-231 cells upon treatment of MCF7 and MDA-MB-231 cells with LINC01929-targeting ASO1 (SEQ ID NO: 1) compared to ASO negative control treatment (SEQ ID NO: 5). Gene ontology (GO) of upregulated genes was established for genes meeting the significance thresholds described in FIG. 10. Biological processes were grouped by common functional pathways.

FIG. 17B shows curated biological processes enriched in genes downregulated in MCF7 and MDA-MB-231 cells upon treatment of MCF7 and MDA-MB-231 cells with LINC01929-targeting ASO1 (SEQ ID NO: 1) compared to ASO negative control treatment (SEQ ID NO: 5). Gene ontology (GO) of downregulated genes was established for genes meeting the significance thresholds described in FIG. 10. Biological processes were grouped by common functional pathways.

FIG. 18 shows 25 curated network of significant immune-, antigen presentation machinery (e.g. TAP proteins, HLA I), and proteasome-related biological processes enriched in upregulated genes upon treatment of MCF7 cells with LINC01929-targeting ASO1 compared to ASO negative control treatment (SEQ ID NO: 5).

FIG. 19 shows a curated network of LINC01929-miRNA-mRNA interactions in MCF7 cells, displaying mRNAs and miRNAs involved in cancer, immune, and antigen presentation processes that are regulated by targeting LINC01929.

FIG. 20A shows the predicted binding sites of miR-215-5p (SEQ ID NO:10) and miR-192-5p (SEQ ID NO: 11) by Incbook 2.0, and their respective positions on the LINC01929 transcripts, which is present in the two longest transcripts (ENST00000589125.5 and ENST00000592405.3) but not the other nine LINC01929 transcripts.

FIG. 20B shows the relative luciferase activity in MCF7 and MDA-MB-231 cells transfected with a pmirGLO dual-luciferase vector containing the predicted LINC01929 target sequence for miR-192-5p (wildtype) (SEQ ID NO:12 forward, SEQ ID NO:13 reverse) or a mutated version (mutant) (SEQ ID NO:14, SEQ ID NO:15) and treated with miR-192-5p mimic or negative control (SEQ ID NO:16) (N=5). Statistical significance was assessed by a two-way ANOVA followed by a Šídak multiple comparison test. Error bars represent the standard deviation. Luciferase activity was normalized to cells treated with the negative control mimic and wildtype sequence vector.

FIG. 21 shows the ceRNA location binding sites of selected miRNAs that were differentially expressed upon treatment of MCF7 and MDA-MB-231 cells with LINC01929-targeting ASO1 (FIG. 18). The position targeted by of the ceRNA sites described herein is shown by labeled vertical lines.

FIG. 22 shows log 2 fold changes in gene expression in MDA-MB-231 cells, as determined by a Affymetrix 2.0ST gene array (N=3) following treatment of LINC01977-targeting ASO (SEQ ID NO: 17), which compared to ASO negative control treatments (SEQ ID NO:5). A significance threshold of p<0.05 and fold-change cutoffs of +1.2311 were applied. Labeled genes represent those of particular interest.

FIG. 23 shows biological processes associated with antigen presentation in genes regulated in MDA-MB-231 cells upon treatment of MDA-MB-231 cells with LINC01977-targeting ASO (SEQ ID NO:17) compared to ASO negative control treatment (SEQ ID NO:5). The analysis includes genes that meet the significance thresholds described in FIG. 16. No antigen presentation-related biological process reached statistical significance among upregulated or downregulated genes.

FIG. 24 shows differentially expressed proteins upon treatment of MCF7 cells with LINC01929-targeting ASO1 (SEQ ID NO: 1) compared to ASO negative control treatment (SEQ ID NO: 5) for 48 h as detected by mass spectrometry proteomics.

FIG. 25 shows the correlation between the transcriptome (FIG. 11) and proteome (FIG. 22) with genes/proteins critical in antigen presentation and immune response regulation identified.

FIG. 26 shows curated biological processes enriched in proteins regulated in MCF7 and upon treatment of MCF7 cells with LINC01929-targeting ASO1 (SEQ ID NO: 1) compared to ASO negative control treatment (SEQ ID NO: 5), reflecting a gene ontology (GO) analysis of regulated processes meeting the significance thresholds.

FIG. 27 shows the representative images and relative fluorescence intensity of cell surface MHC I/HLA I levels in MCF7 and MDA-MB-231 cells following 48 h treatment with LINC01929-targeting ASO1 (SEQ ID NO: 1) and ASO2 (SEQ ID NO: 2) compared to ASO negative control treatment (SEQ ID NO: 5, N=5). Significance determined by a one-way ANOVA followed by Bonferroni multiple comparisons test. Error bars represent the standard deviation.

FIG. 28 shows the representative images and relative fluorescence intensity of cell surface MHC I/HLA I levels in MCF7 and MDA-MB-231 cells following 48 h treatment with siRNA (SEQ ID NO: 8 and SEQ ID NO: 9) compared to negative control siRNA treatments (obtained from IDT, catalog 51-01-14-04).

FIG. 29 shows the Western blot analysis of immunoproteasome subunit PSMB8 levels in MCF7 and MDA-MB-231 cells following 48 h treatment with LINC01929-targeting ASO1 (SEQ ID NO: 1) and ASO2 (SEQ ID NO: 2) compared to ASO negative control treatment (SEQ ID NO: 5, N=5).

FIG. 30 shows the relative proteasomal activity in MCF7 and MDA-MB-231 following 48 h treatment with LINC01929-targeting ASO1 (SEQ ID NO: 1), and compared to ASO negative control treatment (N=7, SEQ ID NO: 5). The activity was detected using the fluorometric Proteasome Activity Assay Kit (Abcam, Cat #ab107921). The proteasome activity was calculated based on manufacturer's protocol and normalized between samples for total protein using Bicinchoninic acid assay. Significance was determined by a one-way ANOVA followed by Dunnett's multiple comparisons test. Error bars represent the standard deviation.

FIG. 31 shows the number and length of peptides from a pan-HLA I pulldown experiment completed on MCF7 cells treated with LINC01929-targeting ASO1 (SEQ ID NO: 1) compared to ASO negative control treatment (N=4, SEQ ID NO: 5).

FIG. 32 shows that targeting LINC01929 with ASO1 (SEQ ID NO:1). compared to ASO negative control treatment (N=5, SEQ ID NO:5) in MCF7 cells induced an overall enrichment of pan-HLA I immunoprecipitated peptides, with 631 peptides upregulated, and only 29 downregulated peptides (p<0.05, log 2 foldchange 0.3 cutoff).

FIG. 33 presents the fold change in peptides predicted or validated to bind HLAs upon treatment with ASO1 in the subset 1256 peptides (NetMHC-predicted binders and IEBD-validated peptides specific to MCF7 HLA I allele).

FIG. 34 illustrates a lack of correlation between the log 2 fold change of immunoprecipitated peptides, and the levels of corresponding log 2 fold change in source protein from the proteomics experiment presented in FIG. 21.

FIG. 35A shows the quality of the upregulated peptides induced by LINC01929-targeting ASO1 as potential cancer-associated antigens capable of inducing an anti-tumor T cell response. IEDB was utilized to query the peptide source proteins for cancer association. Of the upregulated peptides source proteins, 91% have been previously shown to be associated with cancer with 23.5% of them in breast cancer.

FIG. 35B shows a similar analysis to that of FIG. 35A for the 1256 peptides identified in FIG. 28, revealing that out of the 114 upregulated source proteins, 98% of them were previously identified as associated with cancer, with 27.7% of them previously studied in breast cancer.

FIG. 36 shows the upregulation of HLA I presentation of these previously validated tumor-associated antigenic peptides by LINC01929-targeting ASO1 compared with ASO negative control treatment (SEQ ID NO: 5, N=4) occurred in the absence of source protein upregulation.

FIG. 37 shows representative images and relative fluorescence intensity of cell surface PD-L1 levels in MCF7 (N=3) and MDA-MB-231 (N=5) cells following 48 h treatment with LINC01929-targeting ASO1 and ASO2 compared to ASO negative control (SEQ ID NO: 5). Significance was determined by a one-way ANOVA followed by Dunnett's multiple comparisons test.

FIG. 38 shows PBMCs from five different breast cancer patients, who had undergone CD3 CD28 T cell activation (which removes the need for MHC I restriction). PBMCs were co-cultured with MCF7 cells (3:1 ratio of PBMCs to MCF7 cells) treated with either LINC01929-targeting ASO1 (SEQ ID NO: 1), ASO2 (SEQ ID NO: 2), or negative control ASO (SEQ ID NO: 5), in the presence or absence of anti-PD-1. Representative images (top) and quantification of MCF7 cell death percentages (bottom, N=5) are provided. Statistical significance was determined using two-way ANOVA with Tukey's multiple comparisons test. Error bars indicate standard deviation.

FIG. 39 shows representative images (left) and quantification (right) of apoptotic cells 48 hours after treatment with LINC01929-targeting ASO1 (SEQ ID NO: 1), ASO2 (SEQ ID NO: 2), or negative control ASO (SEQ ID NO: 5) in MCF7 and MDA-MB-231 cells (N=4), assessed by flow cytometry. Statistical significance was determined using a one-way ANOVA followed by Dunnett's multiple comparisons test. Error bars represent the standard deviation.

### DETAILED DESCRIPTION OF THE INVENTION

The terms “administration” or “administering” a composition should be understood to mean providing a composition of the present invention to an individual in a form that can be introduced into that individual's body in an amount effective for prophylaxis, treatment, or diagnosis, as applicable. Such forms may include e.g., oral dosage forms, injectable dosage forms, transdermal dosage forms, inhalation dosage forms, and rectal dosage forms.

The term “pharmaceutically acceptable excipient”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

Unless otherwise indicated, a “diagnostically effective amount” of a composition is an amount sufficient to diagnose a disease or condition. In general, administration of a composition for diagnostic purposes does not continue for as long as a therapeutic use of a composition, and might be administered only once if such is sufficient to produce the diagnosis.

Unless otherwise indicated, a “therapeutically effective amount” of a composition is an amount sufficient to treat a disease or condition, or one or more symptoms associated with the disease or condition.

The term “subject” is intended to include living organisms in which disease may occur. Examples of subjects include humans, monkeys, cows, sheep, goats, dogs, cats, mice, rats, and transgenic species thereof.

Parenteral dosage forms can be administered to patients by various routes including subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions. Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants. These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compositions, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof. If desired, and for more effective distribution, the compositions of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Compositions of the invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compositions of the invention, stabilizers, preservatives, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

An effective amount of one of the compositions of the invention can be employed in pure form or, where such forms exist, in pharmaceutically acceptable salt form. Alternatively, the composition can be administered as a pharmaceutical composition containing the composition of interest in combination with one or more pharmaceutically acceptable carriers. It will be understood, however, that the total daily usage of the compositions and compositions of the invention will be decided by the attending physician within the scope of sound medical judgment. The specific effective dose level for any particular patient will depend upon a variety of factors including the disorder being treated and the severity of the disorder; activity of the specific composition employed; the specific composition employed; the age, body weight, general health, sex and diet of the patient; the time of administration, route of administration, and rate of excretion of the specific composition employed; the duration of the treatment; the risk/benefit ratio; drugs used in combination or coincidental with the specific composition employed; and like factors well known in the medical arts. For example, it is well within the skill of the art to start doses of the composition at levels lower than required to achieve the desired therapeutic effect and to gradually increase the dosage until the desired effect is achieved. In some embodiments, the effective amount relates to the amount necessary or sufficient to realize a desired biological effect which is, for example, reducing, stopping the advance of, or reversing the symptoms of cancer.

RNA compositions as described herein may be formulated in lipid nanoparticles, in lipoplex particles, or in other lipid containing carriers to generate serum-stable formulations for intravenous (i.v.) administration. In some embodiments, an RNA composition is mixed with a lipid containing carrier to obtain a pharmaceutically acceptable delivery of the RNA composition. In some embodiments, a lipid solution comprising one or more cationic lipids in ethanol is injected into an aqueous solution, thereby forming a cationic liposome; upon which the RNA composition can be mixed with the cationic liposome, wherein the ligand is directly complexed with, but not chemically conjugated to, the cationic liposome. A wide variety of cationic liposomes are useful in the preparation of the complexes. Published PCT application WO99/25320, incorporated herein by reference, describes the preparation of several cationic liposomes. Examples of desirable liposomes include those that comprise a mixture of dioleoyltrimethylammonium phosphate (DOTAP) and dioleoylphosphatidylethanolamine (DOPE) and/or cholesterol (chol), or a mixture of dimethyldioctadecylammonium bromide (DDAB) and DOPE and/or cholesterol, or a mixture of dimethyldioctadecylammonium bromide (DDAB) and DOPE, or a mixture of dioleoyltrimethylammonium phosphate (DOTAP) and dioleoylphosphatidylethanolamine (DOPE). The ratio of the lipids can be varied to optimize the efficiency of uptake of the therapeutic molecule for the specific target cell type. The liposome can comprise a mixture of one or more cationic lipids and one or more neutral or helper lipids.

“Coding sequence”, “nucleic acid encoding”, and “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means a nucleic acid can mean Watson-Crick (e.g., A-T/U and C-G) or Hoogsteen base pairing between nucleotides or nucleotide analogs of nucleic acid molecules.

“Consensus” or “consensus sequence” as used herein means a polypeptide sequence based on analysis of an alignment of multiple sequences for the same gene from different organisms. Nucleic acid sequences that encode a consensus polypeptide sequence can be prepared. Immunogenic compositions comprising proteins that comprise consensus sequences and/or nucleic acid molecules that encode such proteins can be used to induce broad immunity against an antigen.

“Fragment” as used herein with respect to nucleic acid sequences means a nucleic acid sequence or a portion thereof, that encodes a polypeptide capable of eliciting an immune response in a mammal that cross reacts with an antigen disclosed herein. The fragments can be DNA or RNA fragments selected from at least one of the various nucleotide sequences that encode protein fragments set forth below. Fragments can comprise at least 10%, at least 20%, at least 30%, at least 40%, at least 50%, at least 60%, at least 70%, at least 80%, at least 90%, or at least 95% of one or more of the nucleic acid sequences set forth below. In some embodiments, fragments can comprise at least 20 nucleotides or more, at least 30 nucleotides or more, at least 40 nucleotides or more, at least 50 nucleotides or more, at least 60 nucleotides or more, at least 70 nucleotides or more, at least 80 nucleotides or more, at least 90 nucleotides or more, at least 100 nucleotides or more, at least 150 nucleotides or more, at least 200 nucleotides or more, at least 250 nucleotides or more, at least 300 nucleotides or more, at least 350 nucleotides or more, at least 400 nucleotides or more, at least 450 nucleotides or more, at least 500 nucleotides or more, at least 550 nucleotides or more, at least 600 nucleotides or more, at least 650 nucleotides or more, at least 700 nucleotides or more, at least 750 nucleotides or more, at least 800 nucleotides or more, at least 850 nucleotides or more, at least 900 nucleotides or more, at least 950 nucleotides or more, or at least 1000 nucleotides or more of at least one of the nucleic acid sequences set forth below.

“Identical” or “identity” as used herein in the context of two or more nucleic acids means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. Identity can be performed manually or by using a computer sequence algorithm such as BLAST or BLAST 2.0.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof.

In various diagnostic embodiments, a reference level is used to inform or establish a threshold for diagnosis. A reference level is that of a reference subject or population of subjects which may be a matched control of the same species, gender, ethnicity, age group, smoking status, BMI, current therapeutic regimen status, medical history, or a combination thereof, but differs from the subject being diagnosed or from whom a sample was obtained in that the reference does not suffer from the disease in question or is not at risk for the disease, alternative aspects, the reference expression level(s) of the gene(s), protein(s), RNA, or the expression level(s) or phosphorylation levels protein(s) is/are level(s) of a subject known to have a tumor. In some embodiments, the measured level is compared to both a reference level of a subject known to not have a tumor and a reference level of a subject known to have a tumor. In some embodiments, the reference level is the mean of a population of expression levels for the corresponding gene.

Expression vectors as described herein can be a circular plasmid or a linear nucleic acid. The circular plasmid and linear nucleic acid are capable of directing expression of a particular nucleotide sequence in an appropriate subject cell. The vector can have a promoter operably linked to the antigen encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

Immune checkpoint inhibitors as used herein include, but are not limited to, A2AR, B7-H3, B7-H4, BTLA, CTLA-4, IDO, KIR, LAG3, PD-1, PD-L1, PD-L2, TIM-3, VISTA (protein), TIGIT (T cell Ig and ITIM domain), CD47, and SIRPalpha. In some embodiments, a composition or method of treatment of cancer described herein is administered prior to, alongside, or after administration of an immune checkpoint inhibitor. The sequencing of administration with respect to a standard of care may be selected by a medical practitioner in view of than-current medical practice in cancer.

In certain embodiments, administration of an antisense compound targeted to a given lncRNA results in reduction of that lncRNA's levels by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 99%, or a range defined by any two of these values.

Compositions and method of treatment and diagnosis herein are preferably directed to cancer. In some embodiments, the cancer is a primary or metastatic brain tumor, a breast cancer, a neuroendocrine tumor, a melanoma, a pancreatic cancer, a prostate cancer, a head and neck cancer, an ovarian cancer, a lung cancer, a bladder cancer, a kidney cancer, a liver cancer, a vaginal cancer, a urogenital cancer, a gastric cancer, a colorectal cancer, a cervical cancer, a liposarcoma, an angiosarcoma, a rhabdomyosarcoma, a choriocarcinoma, a retinoblastoma, a multiple myeloma, a non small cell lung cancer (NSCLC), an adenocarcinoma, a squamous cell carcinoma, a large cell carcinoma, a small cell lung cancer, a primary or metastatic breast cancer, an inflammatory breast cancer, a ductal carcinoma in situ (DCIS), an invasive ductal carcinoma (IDC), an invasive lobular carcinoma (ILC), a lobular carcinoma in situ (LCIS), a male breast cancer, a triple negative breast cancer, a molecular subtype of breast cancer, a paget's disease of the nipple, a phyllodes tumor of the breast, a metastatic breast cancer, or combinations of the foregoing.

### Modulators of Long Non-Coding RNA

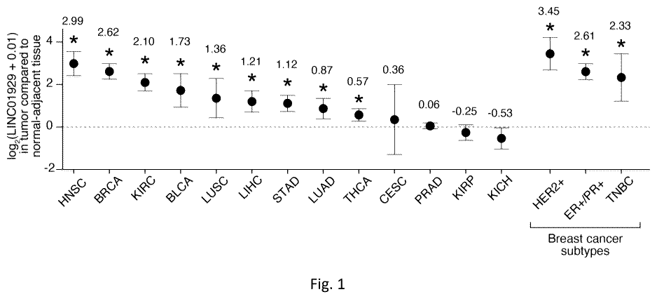

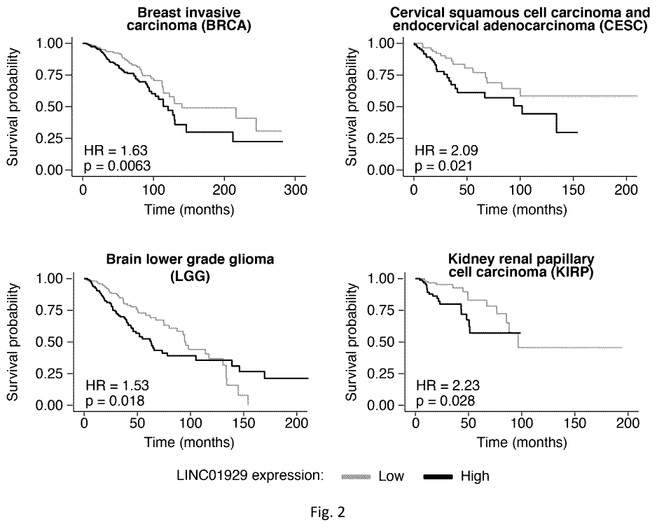

LINC01929 is a long non-coding RNA, located on chromosome 18 and encodes 11 transcripts: ENST00000589125.5 (3067 bases), ENST00000592405.3 (2472 bases), ENST00000774634.1 (1438 bases), ENST00000774630.1 (1198 bases), ENST00000774632.1 (1168 bases), ENST00000774623.1 (1081 bases), ENST00000774628.1 (960 bases), ENST00000657832.1 (948 bases), ENST00000774627.1 (752 bases), ENST00000774625.1 (501 bases) and ENST00000774636.1 (400 bases). The second longest transcript, ENST00000592405.3 (2472 bases) has 2285 bases in common with the longest transcript ENST00000589125.5 (3067 bases), but a difference is the first exon of both transcripts which differs and could result in functional differences. ENST00000589125.5 (3076 bases) is the one LINC01929 transcript reported in NCBI for the LINC01929 gene, https://www.ncbi.nlm.nih.gov/gene/101927229 (NR_110743.1). LINC01929 is highly expressed in many different cancer types and comparatively lowly expressed in normal tissues. Our analysis of LINC01929 across The Cancer Genome Atlas (TCGA) datasets similarly shows high enrichment of expression of the lncRNA across cancers versus normal tissues (FIG. 1). LINC01929 expression has been proposed as prognostic marker for cancer with its expression associated with worse outcomes across cancer types. Our analysis similarly shows that high LINC01929 expression is similarly associated with worse survival in breast, cervical, glioma, and kidney cancers (FIG. 2).

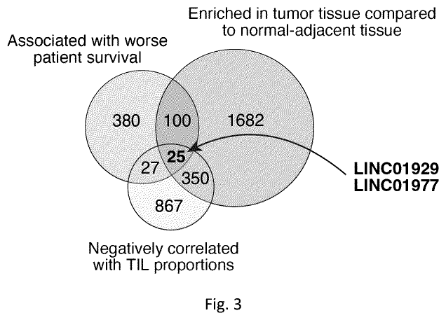

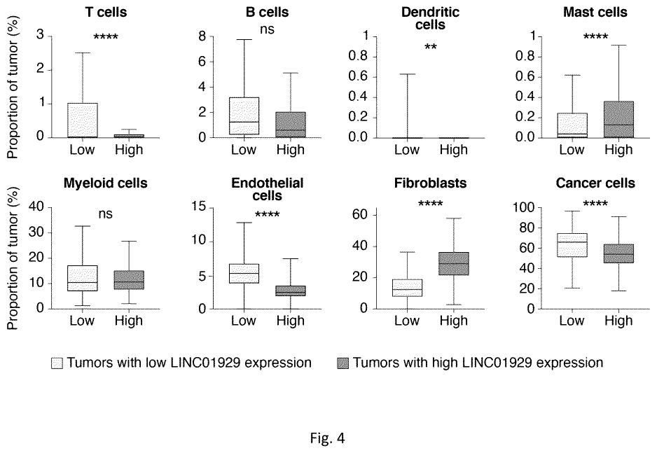

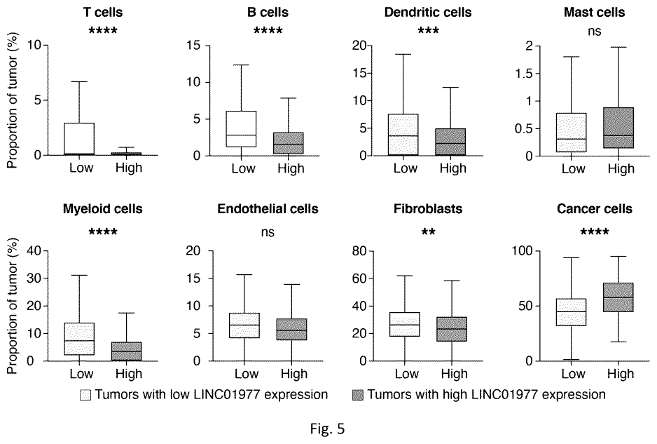

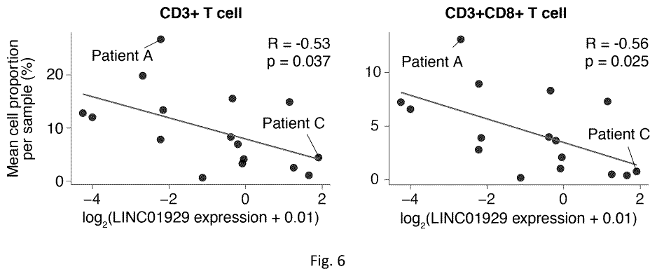

The high expression of LINC01929 has associated has been associated with tumor mutational burden, ICI resistance, and immune cell infiltration across cancer types and it has been proposed to be a biomarker of ICI response. Similarly, we found that LINC01929, along with over 1000 other lncRNAs (e.g., LINC01977) had significant negative correlations with TIL proportions in cancer cohorts (FIG. 3) and some of these also displayed associations with worse survival and tumor tissue enrichment (FIG. 3). Transcriptome deconvolution showed negative associations with T cells and other immune cell populations of LINC01929 (FIG. 4) and LINC01977 (FIG. 5). Our multiplex immunofluorescence staining of breast cancer samples showed that many lncRNAs, including LINC01929 (FIG. 6), expression are negatively correlated with CD3+ and CD3+CD8+ cytotoxic T cell proportions in tumors.

In terms of association with diseases other than cancer, LINC01929 has been reported among a list of genetic loci analyzed for single nucleotide polymorphisms showing associations with Alzheimer's disease and frailty using cross-trait meta-analysis.

In terms of functional activity, LINC01929 was shown to promote the proliferation and/or invasion of oral squamous, bladder, non-small cell lung cancer cells, and breast cancer. In oral squamous, bladder, and non-small cell lung cancer cells, LINC01929 was shown to do this by inhibiting microRNAs (i.e. miR-1179, miR-137-3p, and miR-6875-5p), which target genes that cause cancer growth (i.e. forkhead box C1 protein (FOXC1) and a disintegrin and metalloproteinase with thrombospondin motifs 12 (ADAMTS12)). In breast cancer, LINC01929 was shown to inhibit breast cancer cell cycle progression, cell proliferation, and tumor growth by downregulating the TNF pathway and STAT3 expression. These effects are caused by downregulating LINC01929 using shRNA sequences.

These previously reported effects LINC01929 were not ascribed to a specific transcript and most relevant to the method described here, they do not describe effects of LINC01929 antigen presentation machinery (i.e. HLA I, TAP proteins, and proteasome), presentation of antigens, generation of tumor associated-antigenic peptides, PD-L1 expression, and T cell killing of cancer cells.

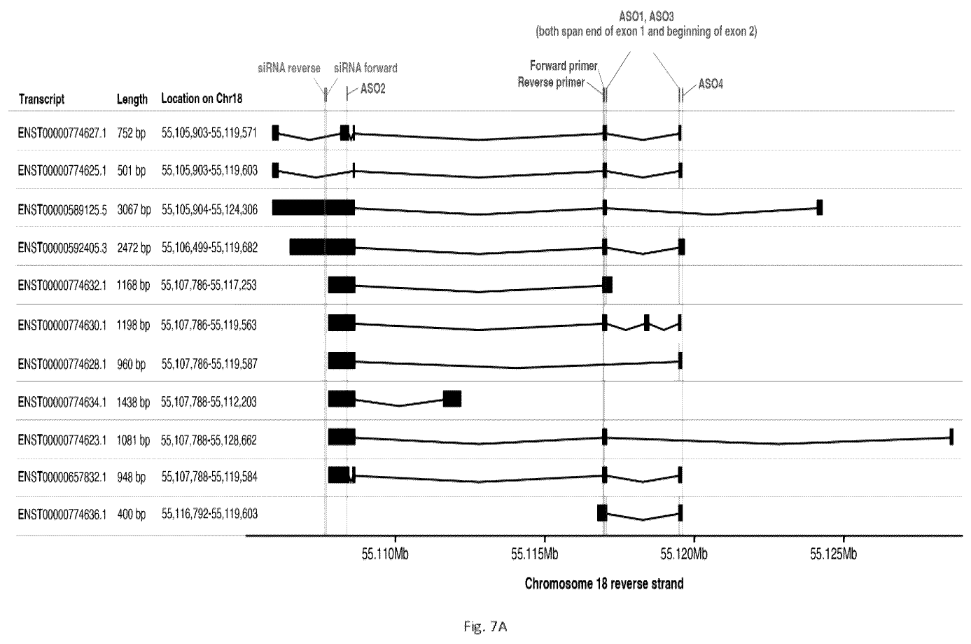

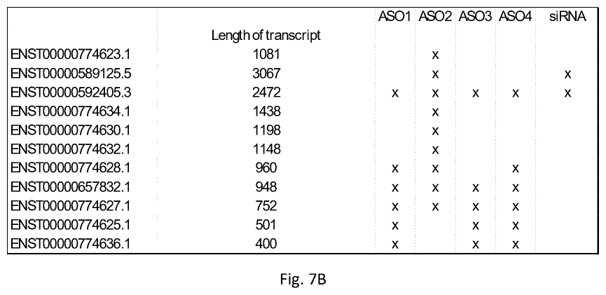

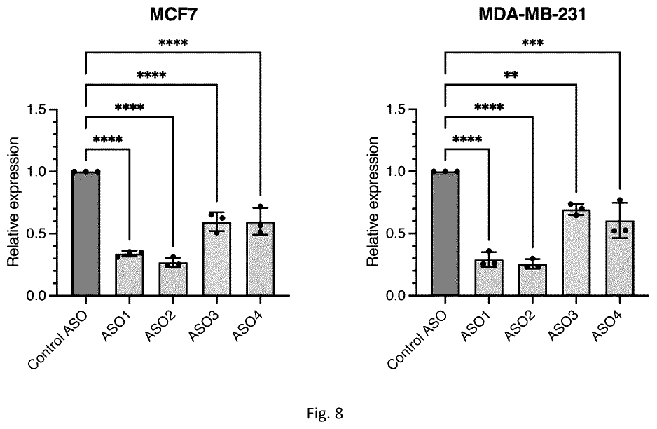

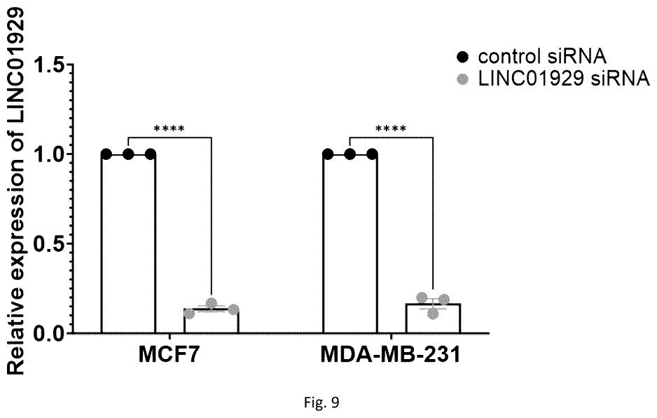

In some embodiments, we describe herein applying antisense oligonucleotides (ASOs) or small interfering (siRNA) that target (e.g., are complementary or approximately complementary to) the RNA sequence of the two longest LINC01929 transcripts (described as ENST00000589125.5 and ENST00000592405.3, FIG. 7) to decrease expression of the transcripts (e.g., by greater than 50%; see FIG. 8 and FIG. 9). The LINC01929 transcripts have both distinct sequences and overlapping sequences; therefore, some of the example ASOs and siRNAs can target multiple transcripts of LINC01929 (FIG. 7). The siRNA sequences only target the two longest LINC01929 transcripts (described as ENST00000589125.5 and ENST0000592405.3), and only ENST0000592405.3 is targeted by all the example ASOs and siRNA sequences. In some embodiments, we provide herein example primers used in quantitative polymerase chain reaction (qpcr) to quantify expression of the LINC01929 transcripts (inclusive of ENST0000592405.3) (Table 1, SEQ ID NO: 6 and SEQ ID NO:7). In some embodiments, ASO sequences are provided that are complementary to the second longest LINC01929 transcript, e.g., as in Table 1 SEQ ID NO: 18:

TABLE 1

| SEQ ID# | Sequence (5′-3′) |
|---|---|
| SEQ ID NO:1 | G* T* C* GTGTCAAGTCA* T* C* |
| SEQ ID NO:2 | C* C* A* TAGAAGAATTA* C* T* |
| SEQ ID NO:3 | G* A* A* GTCGTGTCAAG* T* C* |
| SEQ ID NO 4 | T* G* T* CGTCCACTCTA* A* T* |
| SEQ ID NO:5 | A* A* C* ACGTCTATAC* G* C* |
| SEQ ID NO:6 | TATTCTGGCCCATCGTGGC |
| SEQ ID NO:7 | CTGAAGCAGAGCTCGACCAG |
| SEQ ID NO: 8 | rCrArCrUrCrUrGrArGrArUrArGrArArUrCrUrArArUrUrGGA |
| SEQ ID NO:9 | TCCAATTAGATTCTATCTCAGAGTGAA |
| SEQ ID NO: 10 | AUGACCUAUGAAUUGACAGAC |
| SEQ ID NO: 11 | CUGACCUAUGAAUUGACAGCC |
| SEQ ID NO: 12 | GCTGAGATATCTAGGTCA |
| SEQ ID NO: 13 | TCGATGACCTAGATATCTCAGC |
| SEQ ID NO: 14 | CTTAGCATATGCACGACG |
| SEQ ID NO: 15 | TCGACGTCGTGCATATGCTAAG |
| SEQ ID NO: 16 | UCACCGGGUGUAAAUCAGCUUG |
| SEQ ID NO: 17 | A* C* A* TTGGAAGGATC* A* A* |
| SEQ ID NO: 18 | GCTTTTTTTTTTTTTTTGGTCTCCTAACATCTATTCAAGCTTTTATTTC |
| | ATTTTGATTCTCAGTTCTGTTTCTCAAATTTCAGATCAAGGATGCTTT |
| | AGAAGTAGAAGTAGCCTATTACAACATTCATCTTATGGGCTCCGGGAA |
| | AACACCGGGTGGGGCCAGAAAGCTGAAATGATTTCCCCAAGATGAGAC |
| | AGAGAGTCTCAGAGCCAACCCATGGTTCCTGATTCTAAGACTAGTGCT |
| | CTTGCTATTAAAAAATATTCAACTGTTTCTTTAACTGATTAGCAGAAT |
| | TTATTTCAAGACTGCTTTGCCTAAAATTATCTATAAGACAGTTACATA |
| | AGCCTTTAACTCTAATTTCTTAATACCAATCCAGGATCTTTTCAGTTA |
| | CTGGAGCTTCACTGTACTGCTATCTTTAGGGTACACGCCAATAAAAAA |
| | TATTTGTCAGAGGTTATTTACAAGAAGTTCTTTTGCTACTTTGGTAAT |
| | AAATTATTCTTAGATCCTTAGATCATACGCAGTATGGGCCAACCCTTA |
| | GAGCAATTATCACAACCACAAACTCTTCTAAAAACTGACATAGCACCT |
| | GCTGAGCAAGACTGCCTTAGGAAATTCCTTATCCATTCTTTGGCTACA |
| | ATTCCCTACTACATTTTGGCCTCGGCTAATTGGACCAGAGGGTGAACA |
| | CCTGACCAAAGGCAAACATTCCATTGGCTGGTCAGCATCCTATTAGGA |
| | GGCCTGAAATGAAAGCTTTGACCAATAAAAATCAAGATAACAGGTCCA |
| | ATTAGATTCTATCTCAGAGTGAAAAAGAGTCAAAGTCAACTAGTTTCT |
| | AACAAATGCTGTGATCCACACTTCAGATTGGAGTCTGTGAAATATGCA |
| | CCAACTTCAAGACAATTCCGTGGTAAAACTGGAAAAAATTAGTCATTA |
| | AGACATTAGCCAGGATGTTAGTGGTATGAGAGGATATAAACATGCAGC |
| | CTTGAAATTAAAATACATAGCCTTTAGGCTTTGACCTAGATATCTCAG |
| | CAAAGTGAACAAAGAATAGCATTAAAAGTAAATAAAAGATTGAGGCAG |
| | GAGGATAGCTTGAAGTCAGGAGTTTGAGATTGGCCTGTGCAGTACAGC |
| | GAGACCTCTTCTCTACATAAAAATGTTAAAATTAGATACAAATATAAG |
| | CCCGTAAATAATCTTTGATCTTAAAATTTCTAATTTGGAAAAGTGTCA |
| | TTTGTATGGTGTTCTATATTATTAATGGTTTCAAACTTTAATCAGAGA |
| | TATCTTTCCTTTTCAGGCAAAGCACACACCATGAAAGATACTGCTAAA |
| | AGTTTTTAATATTGCCAGTAATATTATCAAGGTAATTAAAGGGCTTGT |
| | AATAATATTTTGTAATAATATCTGAATATCTTAGTGATTAAACTTGGA |
| | AAAGCCAAACTAGATAGAGAATGACACTGAGAAGAGTAGATAGCACAG |
| | GGTCCAAGGTCAGGCGTGGTTCAACAATTGTCCATAGAAAGTCTTTGT |
| | TTTGATCAGGATATATGAGTTGGAAAATTTCGTGATCGTGGATGAAGT |
| | TTTCTTTGTACTTTCATGAAAAACTACCATTTAAAATTTGGCAGATGT |
| | TTAAAAATGATGATTTTTTTAAAACTACCATACTAGACAATGTCATTT |
| | GTAAATCAATGTGTAATTAAAACATTTTGAAAAGATGCCCATACCAGA |
| | CCTCCAGATTTTCTCCACATTTGAGTAACTTGGAATTTGATTTTCTTC |
| | ATAGCCTGTTAGCCATTTTGACTGAAACAGGACCACACCAAAGGCAAA |
| | GGCTGGTTGTTTCAGTCCAGAGATGTGCCTCCATAGAAGAATTACTTT |
| | GAACAAATCTTAATAGATTTTAGGAAAATATTTTAAAATCAACTGTTG |
| | GAGAATTTAACTATTTTCAGATGAACAGTTTATTGCTTGATTGTTTTT |
| | CCCTTCTTATTCTTCCATGTGGTTTTATGCAGAAGTGATCTGGTTGTA |
| | AAATATGAAGAGATGGCAATAAACTCATTTGGCATTATTTGGATCAAG |
| | CAGCTGGTTGACTTAAATCAGTTATATCACTTCTGTCCACCTCCATGC |
| | GAAGCACTGGTCCAGACCACCATCTCCCTCACCTGGGTTACTACAGCA |
| | GCTCCCAGCTGGTCTCTCTGCTCCTCTTCTCACTTCTCCAATCTCAGA |
| | AAGGACTGGCCTGAAGCAGAGCTCGACCAGGACAGCTTCCTCCCACCA |
| | CACACCAGTCCATCTGTTTCTCATCTGCTGACCATGCCACGATGGGCC |
| | AGAATAGGATTCAGGAGGCTTCTGAAGTCGTGTCAAGTCATCTTTCCT |
| | CAGCTGGATGACTGCGATAGCCTCCTACTGGTTTCCTGTCGTCCACTC |
| | TAATCTCTTTCCCACAGCCACAGTGAACTTTGTCCAAAGGAAATCAGA |
| | TTGTGTACTCCCCTGCTTAAAACCTTCCAATGGTTTTGCATTGCACTC |
| | TAAAAAAAAAAAAAATCTAACTC |

In some embodiments, ASOs are provided that comprise 16 contiguous nucleotides of one of SEQ ID NO: 1, SEQ ID NO: 2, SEQ ID NO: 3, or SEQ ID NO: 4 (Table 1). In some embodiments, including ASO1, ASO2, ASO3, and ASO4 as exhibited herein and in the Fig.s, ASOs are 16 nucleotide sequences with a PS backbone, with LNA modifications. In some embodiments, the sequences contain 10 unmodified DNA nucleotides with the configuration 3-10-3, representing the nucleotide counts of LNA-DNA-LNA regions. Common design patterns for LNA gapmers include configurations such as 3-10-3, 4-8-4, or 3-9-3, representing the nucleotide counts of LNA-DNA-LNA regions.

In other embodiments, example ASOs are provided (e.g., Table 1, SEQ ID NO: 1, SEQ ID NO: 2, SEQ ID NO: 3, or SEQ ID NO: 4) that comprise one or more of the possible modifications to the nucleotide sequences LNAs, peptide nucleic acids (PNAs), phosphorothioate, 5-methylcytosine (5mC), 2′O-methyl or 2′O-methoxyethyl ASOs, morpholino (changes in the furanose ring structure), 2′O-fluorine: used with phosphorothioate and 2′O-methoxyethyl ASO, conjugated with N-acetylgalactosamine, N-acetylgalactosamine: used with phosphorothioate, 2′O-fluorine, and 2′O-methoxyethyl ASO. The examples in Table 1 can contain unmodified DNA nucleotides in the center with the configurations such as 3-10-3, 4-8-4, 4-10-2, or 3-9-3 representing the nucleotide counts of modification-DNA-modification regions.

In some embodiments, an ASO contains phosphorothioate backbone modifications and is bookended by locked nucleic acids in a locked-unlocked-locked configuration e.g., 3-10-3 or 3-9-3. In some embodiments, an ASO is an “Antisense LNA GapmeR” manufactured according to a process generally available through an ASO manufacturer such as Qiagen (www.qiagen.com). Manufacturing and handling instructions found within the Antisense LNA GapmeR Handbook are hereby incorporated by reference.

In another embodiment, example siRNA dsRNA sequences are provided that target LINC01929 (e.g., are complementary or approximately complementary), inclusive of the two longest LINC01929 transcripts, SEQ ID NO: 8, or SEQ ID NO:9) to decrease expression of the LINC01929 transcript (e.g., greater than 50%; see FIG. 9). DsRNAs comprise a sense strand and an antisense strand, and in some embodiments, the antisense strand comprises a region complementary to a region of the two longest LINC01929 transcripts (FIG. 7). In some embodiments, the region of complementarity is less than 25 nucleotides in length and the antisense strand comprises 13 or more contiguous nucleotides. Table 1 provides an example dsRNA SEQ ID NO: 8 and SEQ ID NO:9 that are annealed to each other. In some embodiments, the nucleotides sequences of the annealed dsRNA can contain one or more modification, including 2′-O-methyl(2′Ome), 2′-fluoro (2′F), 2′-O-methoxyethyl(2′OMOE), phosphorothioate (PS), cholesterol conjugation, GalNAc conjugation, polyethylene glycol (PEG) conjugation, unlocked nucleic acid (UNA), bridged nucleic acid (BNA), 2′-4′ constrained ethyl(2′4′CE).

In some embodiments, a synthetic nucleic acid construct is provided that comprises an anti-sense oligonucleotide (ASO), a small interfering RNA (siRNA), a gapmer, a short hairpin RNA (shRNA), a microRNA (miRNA), or a small interfering RNA (siRNA). In certain embodiments, the ASO comprises at least 10, particularly 10 to 33, consecutive nucleotides of the nucleic acid sequence that is at least 90%, at least 91%, at least 92%, at least 93%, at least 94%, at least 95%, at least 96%, at least 97%, at least 98%, at least 99%, or 100% identical to the LINC01929 human gene sequence or to its complementary sequence. In certain embodiments, the ASO comprises 10, 11, 12, 13, 14, 15, 16, 17, 18, 19, 20, 21, 22, 23, 24, 25, 26, 27, 28, 29, 30, 31, 32, or 33 consecutive nucleotides of the nucleic acid sequence that is at least 90%, at least 91%, at least 92%, at least 93%, at least 94%, at least 95%, at least 96%, at least 97%, at least 98%, at least 99%, or 100% identical to SEQ ID NO: 18.

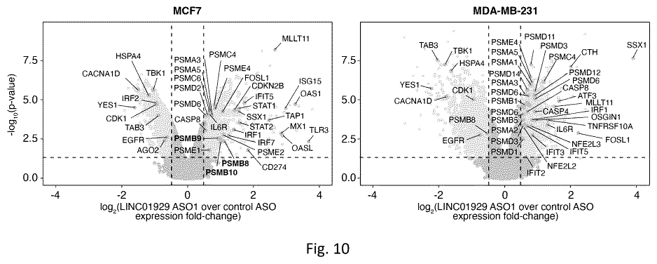

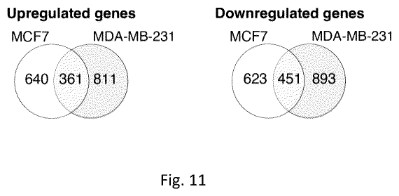

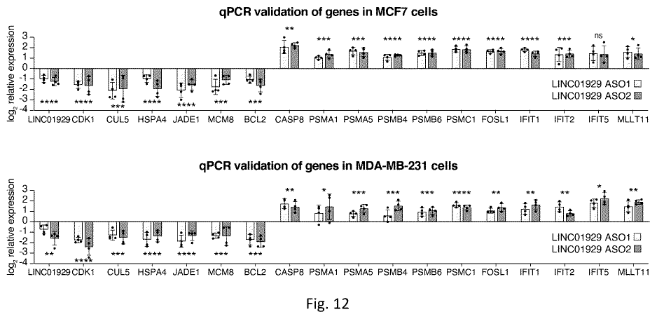

In another embodiment, a method of treating cancer is provided comprising targeting the second longest LINC01929 transcript (ENST00000592405.3) defined in FIG. 7 with a therapy comprising one or more of the active sequences defined in Table 1. We show herein that such targeting results in expression changes (FIG. 10, gene array data) of over 1000 genes in breast cancer MCF7 and MDA-MB-231 cells (FIG. 11), including key genes involved in immune responses against cancer cells and immune responses, such as the interferon induced protein with tetratricopeptide 1 repeats (IFIT, FIG. 10 and FIG. 12).

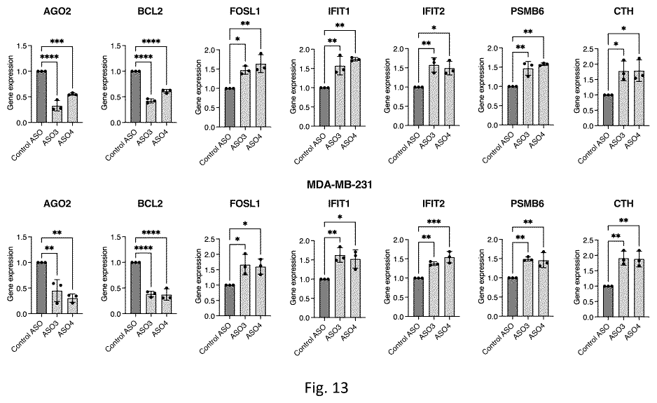

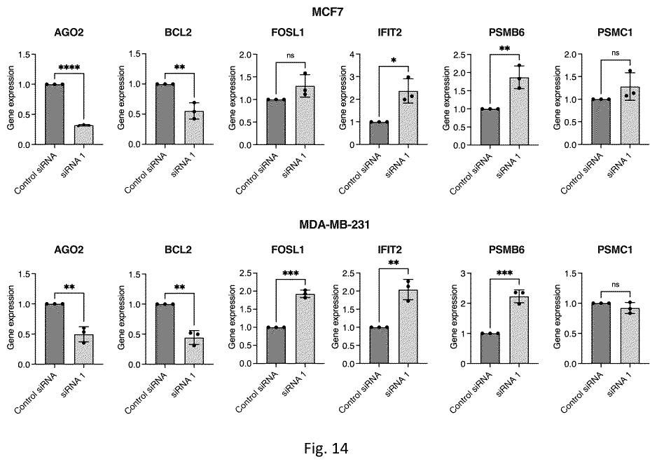

For example, qpcr was performed on a subset of the genes from cells treated with ASOs and siRNAs that target the LINC01929 transcript ENST00000592405.3 (FIGS. 12, 13, and 14). The qPCR results were consistent with the gene array data, showing common upregulation of the key genes involved in immune responses and cancer progression

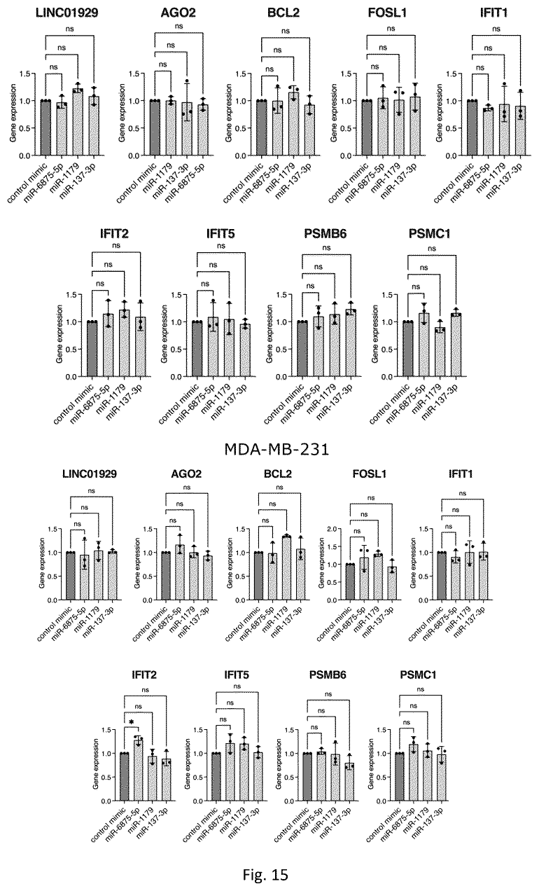

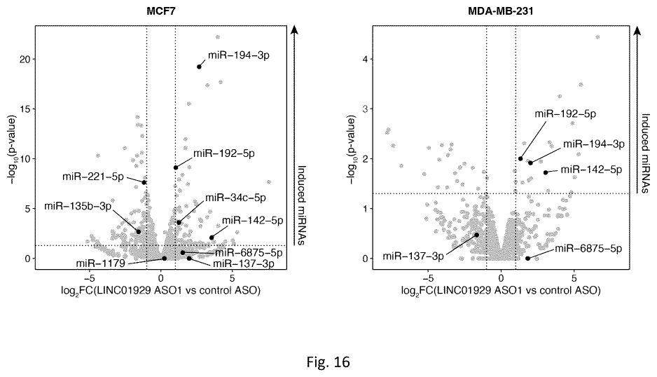

We demonstrate that treating the cancer cells with mimics of the miRNAs miR-179, miR-137-3p, and miR-6875-5p that were previously shown to be regulated by LINC01929, does not induce induction of immune stimulated genes (FIG. 15). Furthermore, small RNA sequencing analysis showed these miRNAs are not induced by targeting the ENST00000592405.3 transcript of LINC01929 with ASO1 (FIG. 16). The therapies and methods described herein are therefore unexpectedly effective in immune induction.

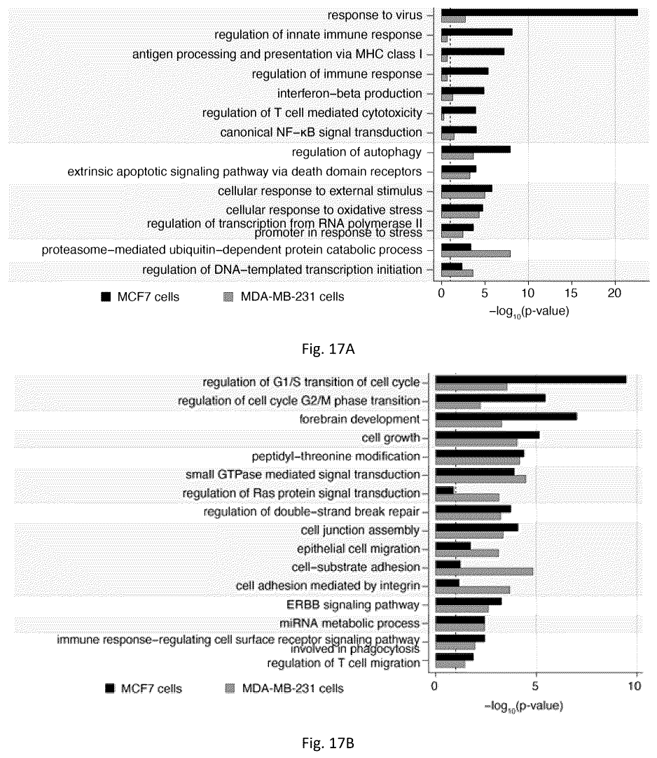

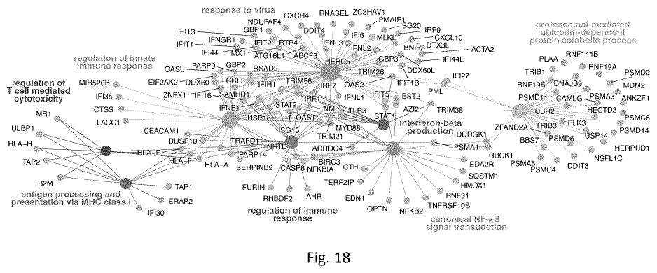

Gene Ontology (GO) enrichment analysis of the upregulated and downregulated genes following LINC01929 targeting in each cell line include those related to extrinsic apoptosis mediation, immune response regulation, and proteasomal subunits (FIG. 17). The most significantly enriched GO terms were among the upregulated genes and included antigen processing and presentation via MHC I, T cell receptor signaling pathway, activation of immune responses and hematopoietic stem cell differentiation (FIGS. 17 and 18).

The GO enrichment analysis highlighted that targeting LINC01929 upregulates the ubiquitin proteasome system (FIG. 17, 18). This could impact various cellular processes, including the immune response such as the generation of tumor associated antigens for presentation to T cells. Overall, the transcriptome data suggest that targeting the LINC01929 transcript ENST00000592405.3 with ASOs or siRNAs increases expression of genes in pathways related to antigen presentation, T cell and immune cell activation (FIGS. 10 to 18).

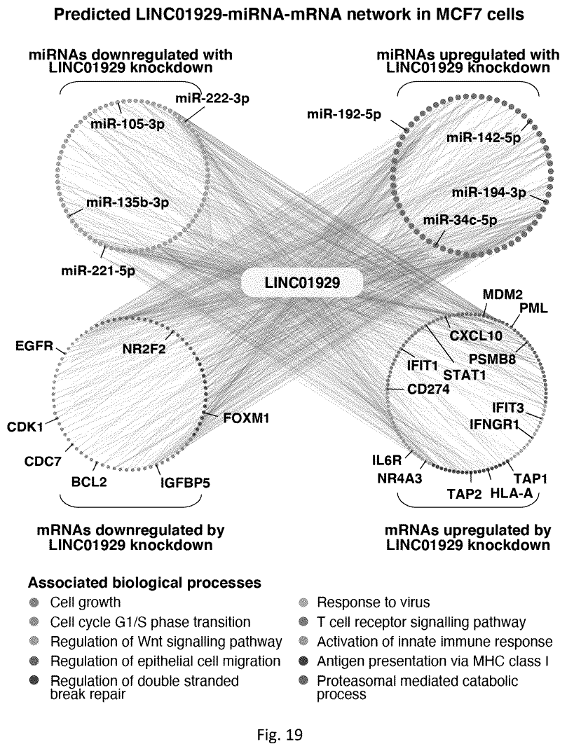

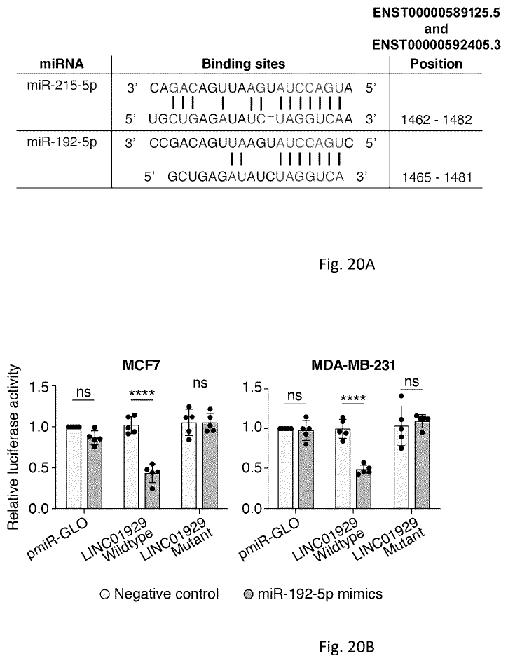

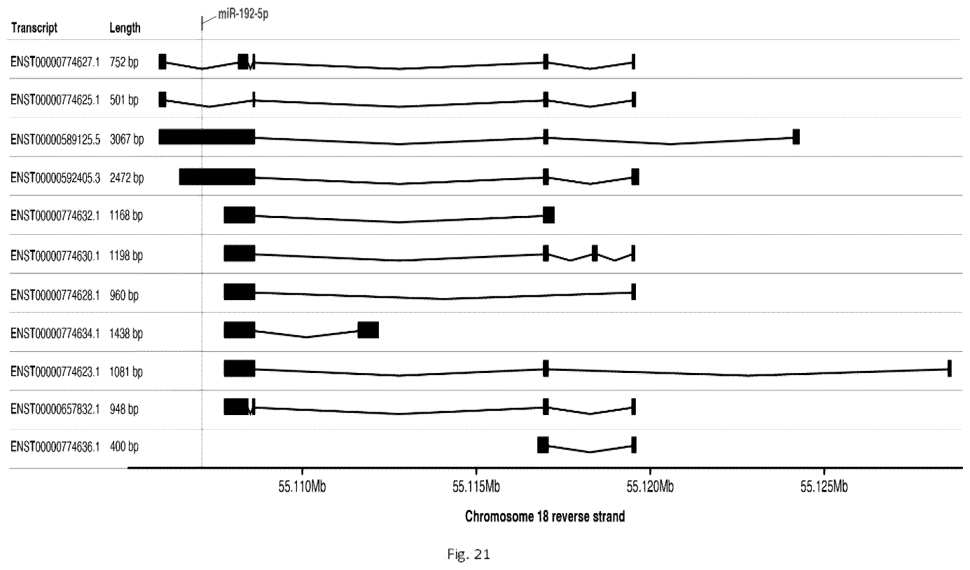

In some embodiments, only the second longest LINC01929 transcript (described as ENST00000592405.3) is targeted with complementary or approximately complementary ASOs or dsRNA, e.g., comprising active sequences defined in Table 1 fully alters expression of miRNAs involved in antigen processing, T cell activation, and proteasomal processes (FIG. 19). These effects on miRNA-mediated mRNA gene expression changes are specific to ceRNA activity (e.g. miR-192-5p, FIG. 20). The ceRNA binding site of miR-192-5p is only present in the two longest LINC01929 transcripts (described as ENST00000589125.5 and ENST0000592405.3) (FIG. 21). Most of these miRNAs regulated by LINC01929 through ceRNA activity are associated with the two longest transcripts of LINC01929. Targeting the other LINC01929 transcripts would not result in the regulation of these miRNAs that are part of the LINC01929-miRNA-mRNA network mediating antigen processing, T cell activation, and proteasomal processes (FIG. 19). Given that ASOs 1, 3, and 4 do not target ENST00000589125.5 (FIG. 7), but ASO1, 3 and 4 are sufficient to induce the immune response effects, it is the unexpected targeting of the second longest transcript (ENST00000592405.3) which fully mediates the effects of induced antigen presentation, proteasomal activation, and T cell activation.

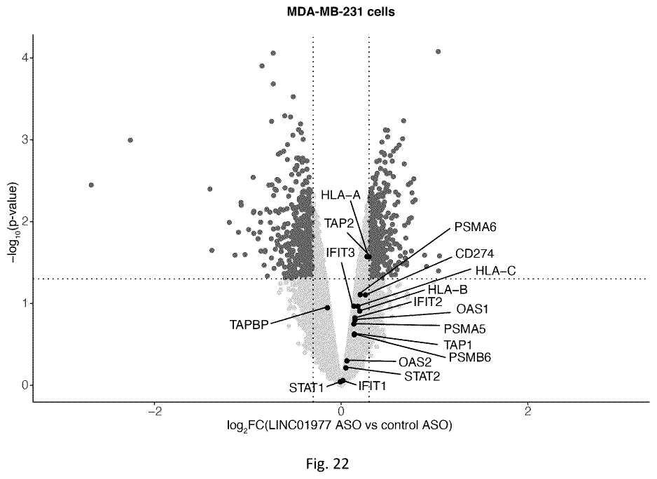

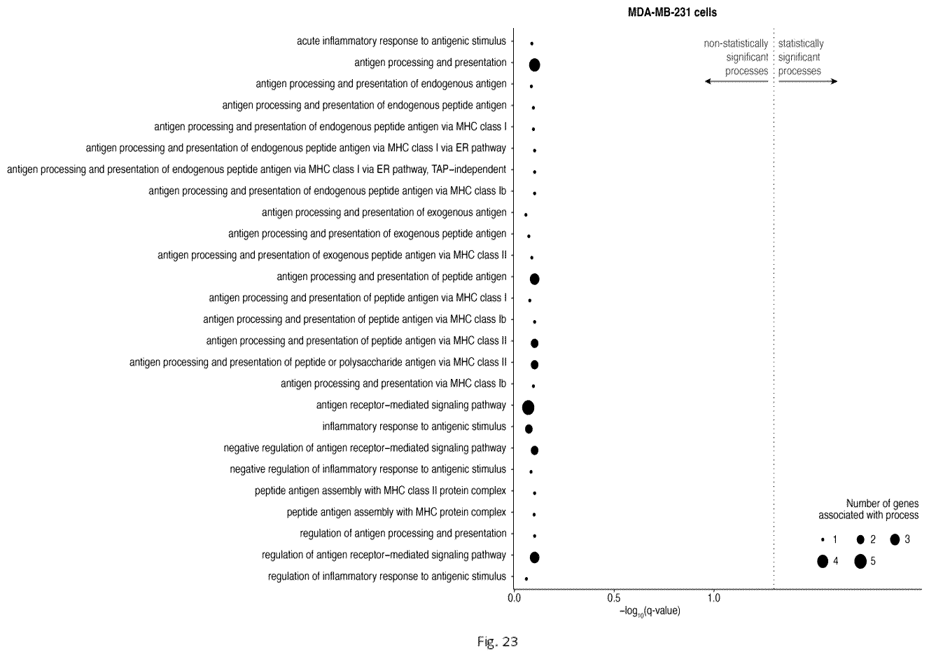

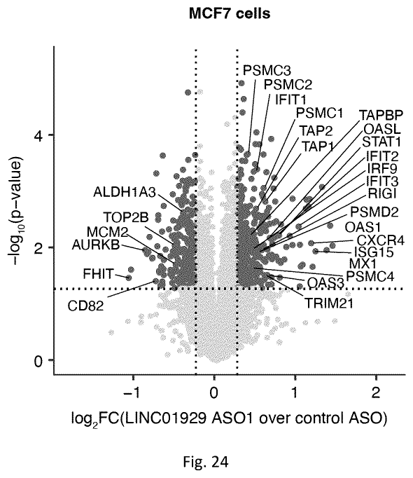

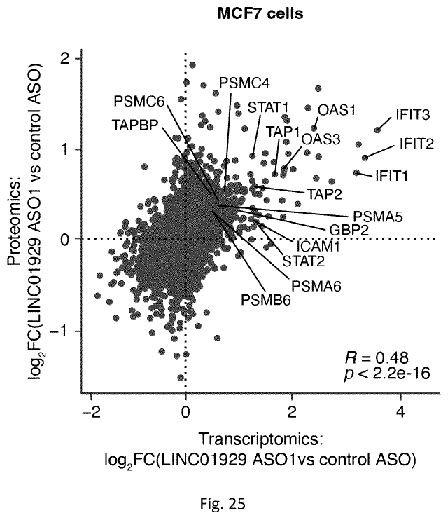

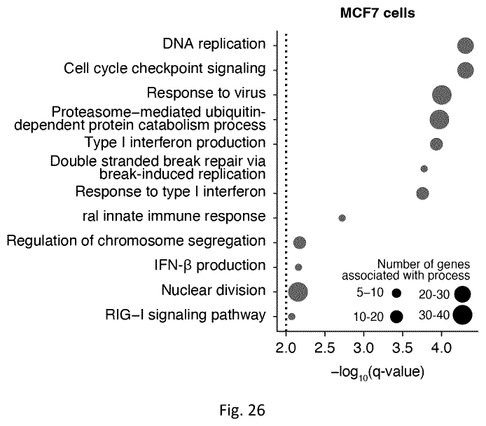

In contrast to targeting the ENST00000592405.3 transcript of LINC01929 with ASOs, targeting LINC01977 with ASOs (another lncRNA that is similarly negatively correlated with T cells in cancer, FIGS. 3, 5), does not induce expression of genes in pathways related to antigen presentation, T cell and immune cell activation (FIG. 22). Instead, GO enrichment of transcriptome data from FIG. 21 shows that targeting LINC01977 induces expression of pathways in other unrelated biological processes (FIG. 23). Targeting the ENST00000592405.3 transcript of LINC01929 results in upregulation of the antigen presentation pathway was further supported by proteomics analysis of MCF7 cells which demonstrated upregulation of these genes at the protein level (e.g. TAP1 and TAP2, TAPBP, the proteasome subunits, IFITs, FIGS. 24 and 25) upon treatment with ASO1 (SEQ ID NO:1). The GO enrichment analysis of the proteomics data similarly supports the effects on these immune pathways (FIG. 26).

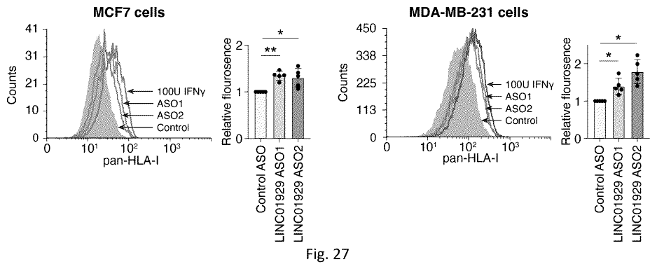

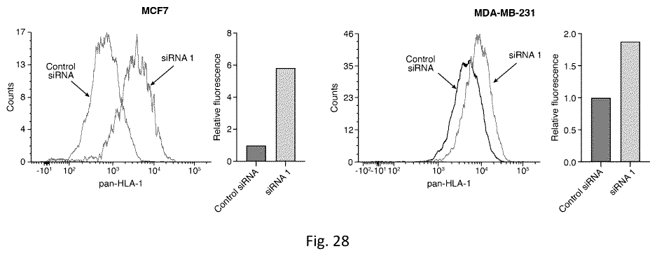

In some embodiments, targeting the LINC01929 transcript ENST00000592405.3 defined in FIG. 7 with complementary or approximately complementary ASO1 and ASO2 or dsRNA sequences (e.g., as defined in Table 1, including SEQ ID NOs: 1-4, 8-9, 18, and selections from 18) results in increased HLA-I/MHC-I on the cell surface. We have demonstrated using flow cytometry analysis that targeting ENST00000592405.3 with ASO1 and ASO2 increased MHC I (i.e., HLA I) on both MCF7 and MDA-MB-231 cells (FIG. 27). This was similarly shown with an siRNA example from Table 1 (FIG. 28) that also targets ENST00000592405.3. In some embodiments, targeting of the second longest LINC01929 transcript (ENST00000592405.3) defined in FIG. 7 with complementary or approximately complementary ASO and siRNA comprising active sequences defined in Table 1 increases immunoproteasome/proteasome activity.

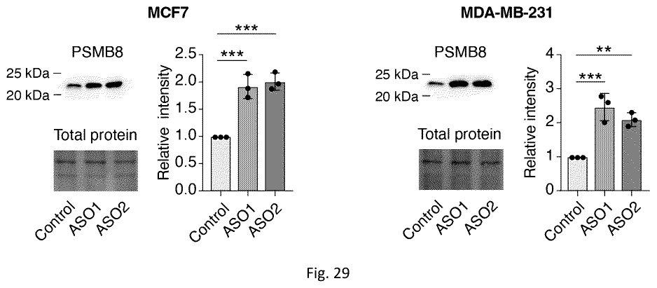

Although constitutively expressed proteasomes contribute to MHC I peptide generation, the immunoproteasome is specialized for this function. Our transcriptome data revealed increased expression of immunoproteasome-specific subunit genes PSMB8, PSMB9 and PSMB10 (FIG. 10), upon targeting the LINC01929 transcript ENST00000592405.3 with ASO1 (SEQ ID NO:1) compared to negative control ASO (SEQ ID NO:5). A western blot confirmed increased PSMB8 (proteasome subunit beta-5i) protein levels in MCF7 and MDA-MB-231 cells (FIG. 29) upon treatment with ASO1 (SEQ ID NO:1) or ASO2 (SEQ ID NO:2).

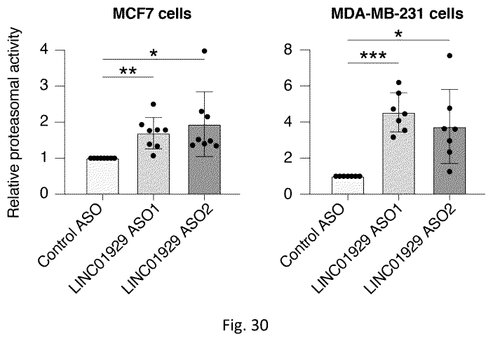

The increased proteasome activity was demonstrated with a fluorometric Proteasome Activity Assay upon treatment with ASO1 (SEQ ID NO:1) or ASO2 (SEQ ID NO:2) (FIG. 30). The observed increased in proteasome activity and cell surface MHC I levels following targeting of the second longest LINC01929 transcript (ENST00000592405.3) suggests enhanced antigen presentation of tumor associated antigen and neo-antigens, allowing enhanced T cell recognition and killing.

In some embodiments, targeting the LINC01929 transcript (ENST00000592405.3) defined in FIG. 7 with complementary or approximately complementary ASOs or dsRNA, e.g., comprising active sequences defined in Table 1 increases the peptides presented on HLA I/MHC I, including tumor-associated antigens.

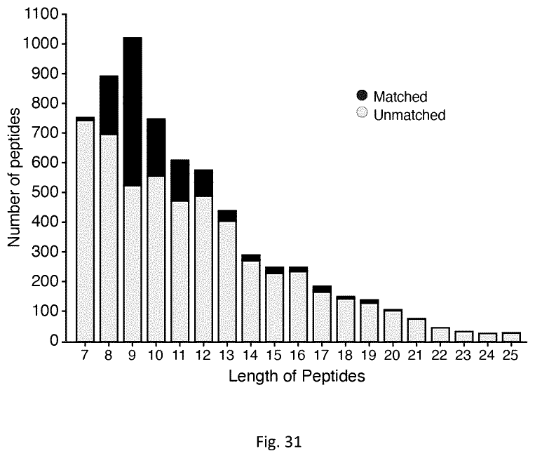

For the HLA immunopeptidome analysis, MCF7 cells were treated with LINC01929 transcript (ENST00000592405.3)-targeting ASO1 (SEQ ID NO:1) compared to negative control ASO (SEQ ID NO:5) and peptides bound to HLA I molecules were immunoprecipitated with a pan-HLA I antibody. A total of 6670 peptides, matching 2032 proteins, ranging from 7-25 amino acids were identified (FIG. 31, Table 2). The abundance distribution of 6670 peptides was centered around the 9-mer length peptides and reflects the length distribution of HLA I-restricted ligands, consistent with a successful HLA I immunoprecipitation. Upregulated peptides identified in the HLA I pulldown appear to be selectively presented with greater efficiency, based on this data. Table 3 further illustrates the finding that among the list of upregulated peptides induced by LINC01929 transcript (ENST00000592405.3)-targeting ASO1 (SEQ ID NO: 1), twenty-five of the epitopes presented by the four HLA I alleles specific to MCF7 cells have been previously reported as tumor associated-antigenic peptides. For example, the epitope ALPPVLTTV from the antigen, La-related protein 1 (LARP1), elicits T-cell response in myeloid leukemia cells.

TABLE 3

| Peptide | Source antigen | HLA Class | Associated cancer |
|---|---|---|---|
| AIVDKVPSV | COPG1 | HLA-A*02:01 | Breast cancer |
| ALPPVLTTV | LARP1 | HLA-A*02:01 | Breast cancer, Leukemia |
| ALWDIETGQQTV | GNB2 | HLA-A*02:01 | Multiple including breast |
| | | | cancer |
| NLMEQPIKV | JUP | HLA-A*02:01 | Multiple including breast |
| | | | cancer |
| PVDLSKWSGPLSL | PEBP1 | HLA-A*02:01 | Breast cancer |
| SLAQYLINV | PCBP2 | HLA-A*02:01 | Multiple including breast |
| | | | cancer |
| SMLGEEIQL | GREB1 | HLA-A*02:01 | Breast cancer, |
| | | | Glioblastoma |
| YLDPSVLSGV | ARL6IP1 | HLA-A*02:01 | Multiple including breast |
| | | | cancer |
| AADAGLLQFL | WASHC2A | HLA-C*05:01 | Melanoma |
| ALWGGPISL | PCNX3 | HLA-A*02:01 | Melanoma, AML |
| ATIGELAQV | MTOR | HLA-A*02:01 | Multiple |
| FADATVEL | SNX1 | HLA-C*05:01 | Melanoma |
| FLITQLKML | MT-ATP8 | HLA-A*02:01 | Melanoma |
| FSNKITPIQSKEAY | AK4 | HLA-A*02:01 | AML |
| FVFPGELL | SLC1A5 | HLA-A*02:01 | AML, Cervical cancer |
| GFGDLKSPAGLQVLND | EEF1B2 | HLA-A*02:01 | Pancreatic cancer |
| IVDNTTMQL | IDH3G | HLA-A*02:01/ | Melanoma, AML |
| | | HLA-C*05:01 | |
| IVNTNVPRASVPDGFLSEL | MIF | HLA-A*02:01 | Pancreatic cancer |
| LEEAFSVF | NONO | HLA-B*18:01 | Pleural cancer |
| LLKGVPFTL | CLIC3 | HLA-A*02:01 | Melanoma |
| LLLDVPTAAVQA | IFI30 | HLA-A*02:01 | Multiple |
| NDHFVKL | GAPDH | HLA-A*02:01 | AML |
| VLADQVWTL | OGG1 | HLA-A*02:01 | Multiple |
| VTVNQSLLSPLVL | KRT8 | HLA-A*02:01 | Pancreatic cancer |
| YLYEVSQLKD | CNDP2 | HLA-A*02:01 | AML |
| FSNKITPIQSKEAY | AK4 | HLA-A*02:01 | AML |

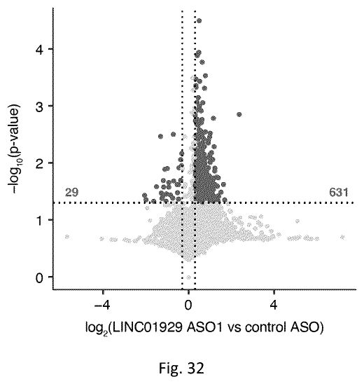

We profiled the 6670 peptides for predicted binding to HLA I with NetMHC pan 4.1 setting the search parameters to peptides specific to the HLA I alleles of MCF7 cells (i.e., A2:01, B18:01/HLA B 44:02, HLA C 05:01) with a minimum rank of 2 (weak binder threshold). This identified 1037 predicted binders. We also profiled the peptides against the Immune Epitope Database (IEDB) for epitopes that have been experimentally validated to bind the HLA I alleles. This revealed 512 previously validated peptides specific to the HLA I alleles found in MCF7, which partially overlapped with the NetMHC-predicted HLA I binders and ranged from 7-mer to 25-mer lengths (FIG. 32). Together, the analyses identified a subset of 1256 peptides of highest interest for further analysis (FIG. 32). Among the remaining 5414 peptides, we identified another 1729 IEBD validated epitopes when we expanded our search parameters to HLA I in general, without allele specificity. This suggests that many of the isolated peptides are potential HLA I binders, or could be non-canonical binders, and therefore we also analyzed the complete set of 6670 immunoprecipitated peptides.

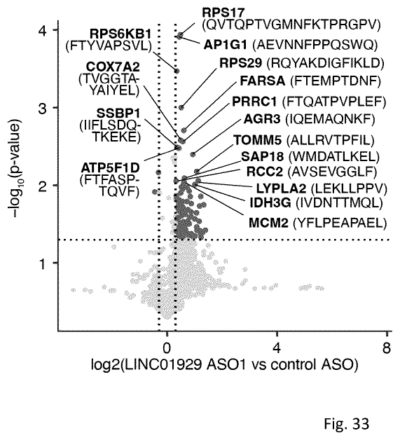

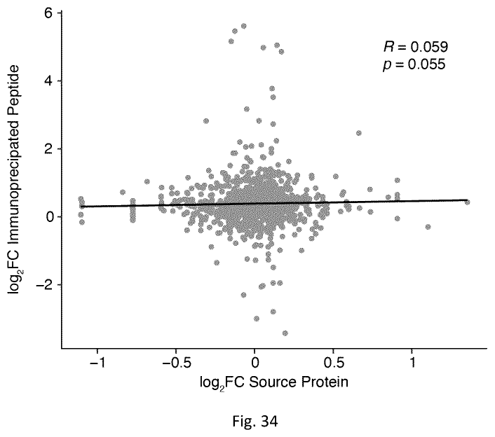

Targeting LINC01929 induced an overall enrichment of peptides, with 631 peptides upregulated, and only 29 downregulated peptides (p<0.05, log 2 foldchange 0.3 cutoff) (FIG. 33). The enrichment in peptides was mirrored in the subset 1256 peptides (NetMHC-predicted binders and IEBD-validated peptides specific to MCF7 HLA I alleles, (FIG. 32). To determine if the peptide enrichment was because of the increased abundance of the source protein or an increase in peptide presentation processes, we analyzed the correlation between the log 2 fold change of immunoprecipitated peptides to the levels of corresponding log 2 fold change in source protein (FIG. 23). The lack of correlation suggests that upregulated peptides in HLA I pulldown are being selectively presented with greater efficiency. Together, this data suggests that targeting the second longest transcript of LINC01929 (ENST00000592405.3) increases antigen presentation.

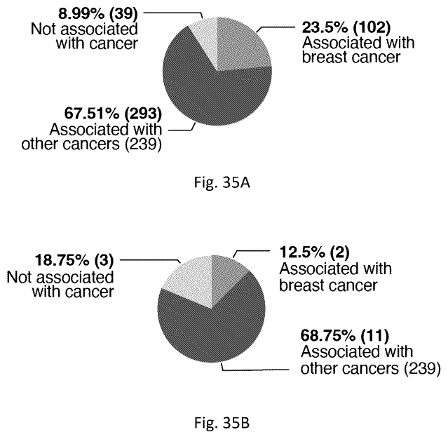

We next assessed the quality of the upregulated peptides as potential cancer-associated antigens capable of inducing an anti-tumor T cell response. We utilized the IEDB to query the peptide source proteins for association with cancer. Of the upregulated peptides source proteins, 91% of have been previously shown to come from source proteins associated with cancer and 23.5% of them in breast cancer (FIG. 35A). A similar analysis for the 1256 peptides identified in FIG. 32, revealed that out of the 114 upregulated source proteins, 98% of them were previously identified as associated with cancer, with 27.7% of them previously studied in breast cancer (FIG. 35B).

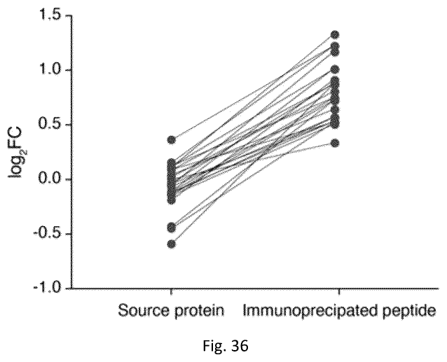

Among the upregulated peptides, 25 are epitopes that have been previously validated as tumor-associated antigenic peptides presented by the HLA I alleles found in MCF7 cells (Table 3). For example, the epitope ALPPVLTTV from the antigen, La-related protein 1 (LARP1), elicits T-cell response in myeloid leukemia cells. The upregulation of HLA I presentation of these previously validated tumor-associated antigenic peptides by targeting the second longest LINC01929 transcript (ENST00000592405.3) with ASO1 (SEQ ID NO:1) occurred in the absence of source protein upregulation (FIG. 36). Our analysis shows that targeting the second longest LINC01929 transcript increases the quantity of the antigens presented via HLA I, including epitopes that should induce anti-tumor immune responses in the tumor microenvironment.

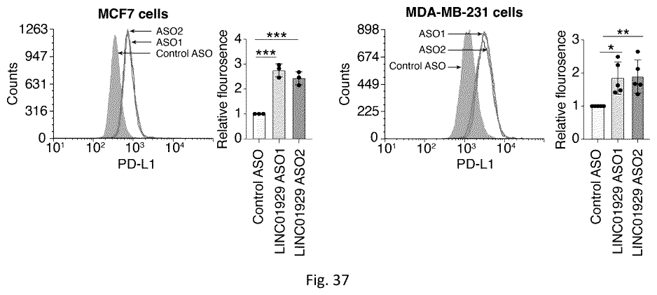

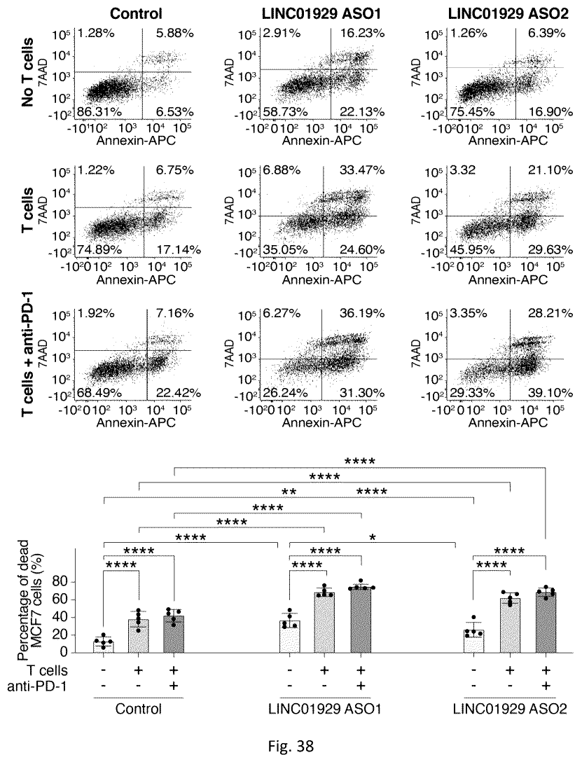

In some embodiments, targeting the second longest transcript of LINC01929 (ENST00000592405.3) with complementary or approximately complementary ASOs or dsRNA, e.g., comprising active sequences defined in Table 1 increases the expression of cell surface PD-L1 on cancer cells. Our transcriptome analysis in MCF7 showed targeting the second longest LINC01929 transcript with an example ASO (ASO1, SEQ ID NO:1) increased the expression of CD274, the gene encoding PD-L1 (FIG. 10). This is in contrast to targeting LINC01977 with an ASO (SEQ ID NO:17), which does not induce expression of CD274/PD-L1 (FIG. 22). Flow cytometry confirmed that PD-L1 cell surface levels were elevated in both MCF7 and MDA-MB-231 cells 48 hours post targeting the second longest LINC01929 transcript with ASO1 (SEQ ID NO:1) and ASO2 (SEQ ID NO:2) (FIG. 37).

In some embodiments, targeting the second longest transcript of LINC01929 (ENST00000592405.3) with complementary or approximately complementary ASOs or dsRNA, e.g., comprising sequences defined in Tables 1 increased killing of cancer cells by T cells. We performed co-culture assays using anti-CD3/CD28 T cell-activated peripheral blood mononuclear (PBMCs) from the blood of 5 different pre-treatment breast cancer patients. MCF7 cells, treated with control or LINC01929 transcript ENST00000592405.3-targeting with ASO1 (SEQ ID NO:1) and ASO2 (SEQ ID NO:2) for 24 hours, were then co-cultured with T-cell activated PBMCs at a 1:3 ratio for an additional 24 hours, with or without anti-PD-1. Flow cytometry analysis revealed increased T cell-mediated killing of the MCF7 cells treated with ENST00000592405.3-targeting ASOs (FIG. 38).

In some embodiments, targeting the second longest transcript of LINC01929 (ENST00000592405.3) with complementary or approximately complementary ASOs or dsRNA, e.g., comprising sequences defined in Tables 1 can be combined with other immunotherapies, including anti-PD-1 and anti-PD-L1 antibodies to increase the killing of cancer cells by CD3/CD28 activated T cells in peripheral blood mononuclear cells (FIG. 38).

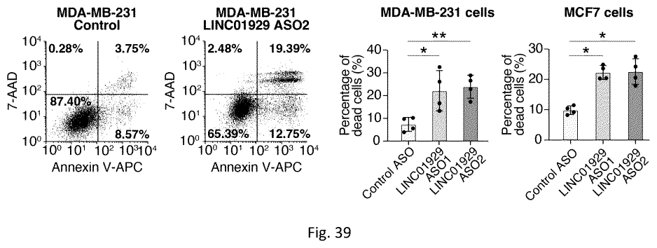

In some embodiments, targeting the second longest transcript of LINC01929 (ENST00000592405.3) with complementary or approximately complementary ASOs or dsRNA, e.g., comprising active sequences defined in Table 1 induces cell death of cancer cells (FIG. 39).

1. A composition comprising a synthetic nucleic acid that encodes a sequence capable of binding to a target sequence, where the complementary target sequence is at least 80% but less than 100% complementary to LINC01929 transcript ENST00000592405.3.

2. The composition of claim 1 in which the nucleic acid is an antisense oligonucleotide.

3. The composition of claim 1 in which the nucleic acid is a double-stranded RNA.

4. The composition of claim 1 in which the nucleic acid comprises RNA, DNA, locked nucleic acid (LNA), morpholino, peptide nucleic acid (PNA), unlocked nucleic acid (UNA), threose nucleic acid (TNA), glycol nucleic acid (GNA), FANA (Fluoro-arabino nucleic acid), or modified RNA selected from 2′-O-methyl(2′Ome), 2′-fluoro (2′F), 2′-O-methoxyethyl(2′OMOE), phosphorothioate (PS), 5-methylcytosine (5mC), cholesterol conjugation, GalNAc conjugation, polyethylene glycol (PEG) conjugation, unlocked nucleic acid (UNA), bridged nucleic acid (BNA), or 2′-4′ constrained ethyl (2′4′CE).

5. The composition of claim 1 in which the composition is an antisense oligonucleotide having a sequence selected from SEQ ID NOs: 1, 2, 3, 4, and 18.

6. The composition of claim 5 in which the composition comprises locked nucleic acid.

7. The composition of claim 1 in which the composition is a short interfering RNA having a sequence selected from SEQ ID NO:8 and SEQ ID NO:9, and SEQ ID 18.

8. The composition of claim 1 further comprising a pharmaceutically acceptable carrier.

9. An expression construct or vector encoding the sequence of a synthetic nucleic acid that encodes a sequence capable of binding to a target sequence, where the complementary target sequence is at least 80% but less than 100% complementary to LINC01929 transcript ENST00000592405.3.

10. A method for treating a cancer in a subject in need thereof, the method comprising administering to the subject at least an effective amount of a therapeutic comprising a synthetic nucleic acid that encodes a sequence capable of binding to a target sequence, where the complementary target sequence is at least 80% but less than 100% complementary to LINC01929 transcript ENST00000592405.3.

11. The method of claim 10 further comprising a first step of administering to the subject a cancer therapy which is a standard of care for the cancer.

12. The method of claim 11 in which the standard of care comprises administration of an immune checkpoint inhibitor agent.

13. The method of claim 10 further comprising, prior to any other steps, identifying the applicability of LINC01929 therapy using previously collected specimens of a subject having cancer, the method comprising mixing labeled probes that are complementary to the second longest LINC01929 transcript (ENST00000592405.3) into the specimen and quantifying the result using an applicable sequencing or amplification paradigm to confirm applicability of LINC01929 transcript ENST00000592405.3 targeting therapy, wherein the therapeutic is administered when the expression level of LINC01929 is greater than a predetermined reference level.

14. The method of claim 10 in which cell surface expression of HLA I/MHC I is increased in cancer cells of the subject.

15. The method of claim 10 in which proteosome activity, optionally including immunoproteasome activity, is induced in the subject.

16. The method of claim 10 in which cancer cells of the subject have HLA I/MHC I presentation such that the number or diversity of immunostimulatory peptides, inclusive of tumor-associated antigens or neoantigens, are increased.

17. The method of claim 10 in which T cells in the subject increase killing of cancer cells of the subject.

18. The method of claim 10 in which cancer cells of the subject are induced to produce PD-L1 on their surfaces.

19. The method of claim 12 in which cancer cells become more susceptible to death by the checkpoint inhibitor agent.

20. The method of claim 10 in which the cancer is selected from a primary or metastatic brain tumor, a breast cancer, a neuroendocrine tumor, a melanoma, a pancreatic cancer, a prostate cancer, a head and neck cancer, an ovarian cancer, a lung cancer, a bladder cancer, a kidney cancer, a liver cancer, a vaginal cancer, a urogenital cancer, a gastric cancer, a colorectal cancer, a cervical cancer, a liposarcoma, an angiosarcoma, a rhabdomyosarcoma, a choriocarcinoma, a retinoblastoma, a multiple myeloma, a primary or metastatic breast cancer, an inflammatory breast cancer, a Ductal Carcinoma In Situ (DCIS), invasive ductal carcinoma (IDC) including IDC Type: Tubular Carcinoma of the Breast, IDC Type: Medullary Carcinoma of the Breast, IDC Type: Mucinous Carcinoma of the Breast, IDC Type: Papillary Carcinoma of the Breast, IDC Type: Cribriform Carcinoma of the Breast, as well as Invasive Lobular Carcinoma (ILC), Lobular Carcinoma In Situ (LCIS), Male Breast Cancer, Molecular Subtypes of Breast Cancer, Paget's Disease of the Nipple, Phyllodes Tumors of the Breast, Triple Negative Breast Cancer, and a Metastatic Breast Cancer.
